(19) United States
(12) Patent Application Publication
(10) Pub. No.: US 2025/0179024 A1
(43) Pub. Date: Jun. 5, 2025

(54) AMYLOID AND ASSOCIATED PATHOLOGY MODULATORS AND METHODS THEREOF

(71) Applicant: Jawaharlal Nehru Centre for Advanced Scientific Research, Bangalore (IN)

(72) Inventors: Govindaraju THIMMAIAH, Bangalore (IN); Madhu RAMESH, Bangalore (IN); Chenikkayala BALACHANDRA, Bangalore (IN)

(73) Assignee: Jawaharlal Nehru Centre for Advanced Scientific Research, Bangalore (IN)

(21) Appl. No.: 18/843,676

(22) PCT Filed: Mar. 3, 2023
(86) PCT No.: PCT/IN2023/050201

§ 371 Date: Sep. 3, 2024

(30) Foreign Application Priority Data
Mar. 4, 2022 (IN) 202241011941

(51) Int. Cl. C07D 221/14; A61K 31/473; A61P 25/28; C07D 401/14

(52) U.S. Cl. CPC C07D 221/14; A61K 31/473; A61P 25/28; C07D 401/14

(57) ABSTRACT

The present disclosure provides a compound of Formula I which are naphthalene monoimide compounds, and process of preparing thereof. The compounds of the present disclosure provide reversal of cognitive decline or improvement of cognitive decline. The compounds of Formula (I) of the present disclosure synergistically modulate metal independent and dependent amyloid toxicity, scavenge reactive oxidative species (ROS), alleviate oxidative stress, emulate Nrf2 mediated stress response, mitochondrial damage, microglial activation and neuroinflammation.

### FIELD OF INVENTION

The subject matter disclosed herein relates to compounds of Formula (I), its stereoisomers or a pharmaceutically acceptable salt thereof. The subject matter in particular relates to naphthalene monoimide (NMI) compounds of Formula (I) its stereoisomers or a pharmaceutically acceptable salt thereof, which are amyloid and associated pathology modulators. The subject matter further relates to a field of drugs for treating the disorders related to the nervous system and in particular relates to a pharmaceutically active compound for use in the treatment of neurodegenerative diseases and neuroinflammatory disorders.

### BACKGROUND OF THE INVENTION

Alzheimer's Disease (AD) is a progressive neurodegenerative disorder that contributes to 60-80% of all forms of dementia affecting more than 55 million people worldwide. Clinically, AD is characterized by the accumulation of amyloid plaques in the brain, and memory and cognitive impairment. AD is a devastating neurodegenerative disorder resulting in major health issues and socio-economic burden worldwide.

The aetiology of AD includes amyloid β (Aβ) amyloidosis, metal ion dyshomeostasis, reactive oxygen species (ROS), oxidative stress, mitochondrial damage, and neuroinflammation. The existing therapeutic molecules for AD only target the symptoms of the disease. There are no approved drugs that target the root cause of AD pathology. Most of the drugs target Aβ, secretases, metals, cholinergic system, ROS independently. Most of the drugs developed are peptides/peptidomimetics, natural products, antibody based therapeutics which face limitations like being prone to degradation, poor bioavailability, and toxicity. The small molecules developed so far were designed to target only one or two targets limiting its ability to alleviate multifaceted AD toxicity.

Decades of research in developing therapeutics aiming at individual disease targets yielded limited successes with no fully approved drugs for AD treatment. The failure of the drug discovery is attributed to the multifactorial nature of AD which needs multifunctional drug molecules. There have been numerous efforts made to develop small molecules targeting two or more pathological factors to tackle multifaceted toxicity. Most of the drugs developed so far target majorly Aβ pathway to treat AD. The recent understanding of the pathobiology of AD revealed the major role of mitochondrial damage and neuroinflammation in AD development and progression. There are limited small molecules targeting multiple etiologies of AD with a multipronged approach.

Hence there is a lack of strategic design of active drug molecules with functional pharmacophores units to address the multiple etiologies of AD. There is a dearth to design and develop synthetic small molecules by integrating multiple pharmacophores to target multiple pathologies including mitochondrial damage and neuroinflammation to synergistically alleviate AD pathology.

### SUMMARY OF THE INVENTION

In a first aspect of the present disclosure, there is provided a compound of Formula (I),

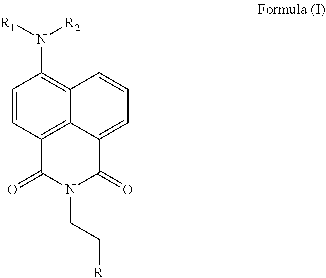

its stereoisomers or pharmaceutically acceptable salts thereof,
wherein R is selected from C₆₋₁₀ aryl, C₅₋₁₈ heteroaryl, or NR₃R₄, wherein C₆₋₁₀ aryl is optionally substituted with two or more hydroxyl groups; R₁ and R₂ are independently selected from C₁₋₆ alkyl, C₂₋₆ alkenyl, or C₂₋₆ alkynyl, wherein C₁₋₆ alkyl is optionally substituted with one or more groups selected from C₆₋₁₀ aryl, C₁₋₁₈ heteroalkyl, C₅₋₁₈ heteroaryl, C₃₋₁₂ cycloalkyl, and C₃₋₁₂ heterocyclyl; and R₃ and R₄ are independently C₁₋₆ alkyl, wherein C₁₋₆ alkyl is optionally substituted with one or more groups selected from C₆₋₁₀ aryl, C₁₋₁₈ heteroalkyl, C₅₋₁₈ heteroaryl, C₃₋₁₂ cycloalkyl, and C₃₋₁₂ heterocyclyl.

In second aspect of the present disclosure, there is provided a process for preparation of compound of Formula (I), its stereoisomers or pharmaceutically acceptable salts thereof, the process comprising: a) reacting a compound of Formula II with Formula III in the presence of a base and a solvent, to obtain a compound of Formula (I)

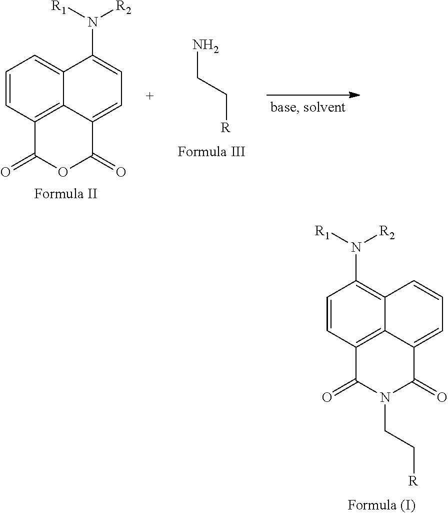

wherein R is selected from C₆₋₁₀ aryl, C₅₋₁₈ heteroaryl, or NR₃R₄, wherein C₆₋₁₀ aryl is optionally substituted with two or more hydroxyl groups; R₁ and R₂ are independently selected from C₁₋₆ alkyl, C₂₋₆ alkenyl, or C₂₋₆ alkynyl, wherein C₁₋₆ alkyl is optionally substituted with one or more groups selected from C₆₋₁₀ aryl, C₁₋₁₈ heteroalkyl, C₅₋₁₈ heteroaryl, C₃₋₁₂ cycloalkyl, and C₃₋₁₂ heterocyclyl; and R₃ and R₄ are independently C₁₋₆ alkyl, wherein C₁₋₆ alkyl is optionally substituted with one or more groups selected from C₆₋₁₀ aryl, C₁₋₁₈ heteroalkyl, C₅₋₁₈ heteroaryl, C₃₋₁₂ cycloalkyl, and C₃₋₁₂ heterocyclyl.

In third aspect of the present disclosure, there is provided a pharmaceutical composition comprising the compound of Formula (I), its stereoisomers or a pharmaceutically acceptable salt thereof with a pharmaceutically acceptable carrier, optionally in combination with one or more other pharmaceutical formulations.

In fourth aspect of the present disclosure, there is provided a method for the treatment of a condition mediated by a neurodegenerative disease or a neuroinflammatory disorder, said method comprising administering to a subject an effective amount of the compound of Formula (I), its stereoisomers or a pharmaceutically acceptable salt thereof, or the pharmaceutical composition comprising the compound of Formula (I), its stereoisomers or a pharmaceutically acceptable salt thereof, with a pharmaceutically acceptable carrier, optionally in combination with one or more other pharmaceutical formulations.

In fifth aspect of the present disclosure, there is provided a method of treatment of a condition mediated by a neurodegenerative disease or a neuroinflammatory disorder, said method comprising administering a combination of the compound of Formula (I), its stereoisomers or a pharmaceutically acceptable salt thereof, or the pharmaceutical composition comprising the compound of Formula (I), its stereoisomers, or a pharmaceutically acceptable salt thereof, with other clinically relevant immune modulator agents to a subject in need of thereof.

In sixth aspect of the present disclosure, there is provided a use of the compound of Formula (I), its stereoisomers or a pharmaceutically acceptable salt thereof, or the pharmaceutical composition comprising the compound of Formula (I), its stereoisomers or a pharmaceutically acceptable salt thereof with a pharmaceutically acceptable carrier, optionally in combination with one or more other pharmaceutical formulations for treatment of a condition mediated by aggregation of one or more protein selected from Aβ42, tau, α-syn, polyglutamine and amylin (IAPP).

In seventh aspect of the present disclosure, there is provided a use of the compound of Formula (I), its stereoisomers or a pharmaceutically acceptable salt thereof, or the pharmaceutical composition comprising the compound of Formula (I), its stereoisomers or a pharmaceutically acceptable salt thereof, with a pharmaceutically acceptable carrier, optionally in combination with one or more other pharmaceutical formulations with other clinically relevant agents or biological agents for treatment of a condition mediated by aggregation of one or more protein selected from Aβ42, tau, α-syn, polyglutamine, and amylin (IAPP).

These and other features, aspects, and advantages of the present subject matter will be better understood with reference to the following description. This summary is provided to introduce a selection of concepts in a simplified form. This summary is not intended to identify key features or essential features of the claimed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts A) dot blot and its quantification of Aβ (10 μM) aggregation inhibition by NMIs (20 μM); B) native gel electrophoresis and western blot for Aβ (10 μM) aggregation inhibition by NMIs (20 μM), C) AFM images of Aβ (10 μM) sample incubated with NMIs (20 μM) for aggregation inhibition study (scale bar 1 m); D) dot blot and its quantification of Aβ aggregates (10 μM) dissolution by NMIs (20 μM), E) AFM images of Aβ (10 μM) with Cu and F) Zn treated with NMIs (20 μM) for aggregation inhibition assessment (scale bar 1 m), in accordance with an implementation of the present disclosure.

FIG. 2 depicts A) dot blot analysis and B) native PAGE (Polyacrylamide gel electrophoresis) and western blot analysis of Aβ (10 μM) aggregation inhibition by M3 at different molar ratio, in accordance with an implementation of the present disclosure.

FIG. 3 depicts dot blot analysis to assess Aβ (10 μM) oligomer inhibition by NMIs (20 μM) and quantification, in accordance with an implementation of the present disclosure.

FIG. 4 depicts A) dot blot analysis and quantification of Aβ aggregates (10 μM) dissolution by NMIs (20 μM), B) native gel electrophoresis and western blot for Aβ aggregates (10 μM) dissolution by NMIs (20 μM), C) AFM images of Aβ aggregates (10 μM) incubated with NMIs to assess dissolution (20 μM) of preformed fibrils, in accordance with an implementation of the present disclosure.

FIG. 5 depicts uv-visible absorption spectra of M1 to M4 (20 μM) with increasing concentration of A) Cu and B) Zn (2.5 to 40 μM), in accordance with an implementation of the present disclosure.

FIG. 6 depicts fluorescence emission spectra of M1 to M4 (20 μM) upon titration with increasing concentration of A) Cu (II) and B) Zn (II) (2.5 to 40 μM), in accordance with an implementation of the present disclosure.

FIG. 7 depicts A) to D) fold absorbance change of M1 to M4 (20 μM), respectively, upon addition of different physiological relevant metals (40 μM), in accordance with an implementation of the present disclosure.

FIG. 8 depicts native gel electrophoresis and western blot for A) Cu and B) Zn-dependent Aβ (10 μM) aggregation and aggregation inhibition by NMIs (20 μM), in accordance with an implementation of the present disclosure.

FIG. 9 depicts A) DPPH (2, 2-diphenyl-1-picrylhydrazyl) radicle scavenging and B) neuronal cell rescue from ROS (reactive oxygen species) damage by NMIs, C) immunofluorescence images Nrf2 (nuclear factor erythroid 2-related factor 2) protein and nuclear stain with DAPI (4′,6-diamidino-2-phenylindole) (Scale bar 5 m), in accordance with an implementation of the present disclosure.

FIG. 10 depicts A) ABTS (2, 2′-azino-bis(3-ethylbenzothiazoline-6-sulfonic acid)) radical scavenging activity of multifunctional modulators at different concentration. B) EC50 values (μM) of M1, M3 and ascorbate from DPPH and ABTS antioxidant assay, in accordance with an implementation of the present disclosure.

FIG. 11 depicts A) to D) assessment of cytotoxicity of multifunctional modulators M1, M2, M3 and M4, respectively, in SHSY-5Y cell by MTT assay, in accordance with an implementation of the present disclosure.

FIG. 12 depicts A) cell viability of SHSY-5Y cells incubated with Aβ (10 μM) and with modulators (20 μM), B) concentration dependent cell rescue from Aβ (10 μM) toxicity by M3, C) cell rescue from Cu (20 μM), and D) Zn (20 μM) dependent Aβ (10 μM) toxicity by modulators (10 μM), in accordance with an implementation of the present disclosure.

FIG. 13 depicts cell rescue from metal dependent-AP toxicity by M3 (50 μM), in accordance with an implementation of the present disclosure.

FIG. 14 depicts A) rescue of Aβ (20 μM) induced disruption of MMP (mitochondrial membrane potential) by M3 in concentration dependent manner, B) quantification of mitochondrial ROS in Aβ treated cells and rescue by M3, C) fluorescence images of mitochondria stained with mito-TG (a mitochondrial probe) shows segmented and abnormal mitochondria in Aβ treatment and rescue by M3 (white arrows), D) western blot analysis and quantification of Cyt c levels in Aβ and along with M3 treated cells, E) fluorescence images of cells stained with DAPI for nucleus visualization, induction of apoptotic features by Aβ treatment (white arrows) compared to control (i to iv represent normal, abnormal shape, nuclear shrinkage, and chromatin condensation, respectively, in the inset figures) and rescue by M3 (scale bar 50 m), F) quantification of percentage apoptotic cells, in accordance with an implementation of the present disclosure.

FIG. 15 depicts A) fluorescence images of SH-SY5Y cells treated with Aβ (20 μM) alone and with M3 (20 μM), stained with Mitotracker orange, B) fluorescence images of mitochondrial ROS generated by Aβ (40 μM) in SHSY5Y cells and its amelioration by M3 (50 μM) (Scale bar 25 μM) stained with MitoSox, in accordance with an implementation of the present disclosure.

FIG. 16 depicts A) bio-AFM (atomic force microscopic) characterization of microglial activation and its attenuation by M3, overlay of confocal microscopy images (DAPI for nucleus, Iba1 and actin with blue, green and red colour labelled, respectively) and AFM height image (scale bar 20 μM), the dashed line represents the morphological features of normal (ramified) activated state (amoeboid) and rescue by M3, B) mean fluorescence intensity quantification of Iba1 (ionized calcium-binding adapter molecule 1) (>150 cells used for quantification), C) to E) quantification of height, log DMT (Derjaguin, Muller, Toropov) modulus and adhesive force respectively, F) western blots of NF-kβ, TNFα and IL6 with R Actin as control (Ctl), G) quantification of NF-kβ, TNFα and IL6 levels relative to control (n=4), in accordance with an implementation of the present disclosure.

FIG. 17 depicts immunofluorescence images of microglial activation by Aβ and its reduction by M3 (scale bar 20 μM), in accordance with an implementation of the present disclosure.

FIG. 18 depicts fluorescence images of actin labelling for microglial cells of control cells and Aβ alone and with M3 (scale bar 10 μM), in accordance with an implementation of the present disclosure.

FIG. 19 depicts AFM height (3D projection), log DMT and adhesive force images, in accordance with an implementation of the present disclosure.

### DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “at least one” used herein refers to one or more and thus includes individual components as well as mixtures/combinations.

The term “pharmaceutically acceptable salt” embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methane sulphonic, ethane sulphonic, benzene sulphonic or p-toluene sulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

Other preferred salts according to the invention are quaternary ammonium compounds wherein an equivalent of an anion (X−) is associated with the positive charge of the N atom. X− may be an anion of various mineral acids such as, for example, chloride, bromide, iodide, sulphate, nitrate, phosphate, or an anion of an organic acid such as, for example, acetate, maleate, fumarate, citrate, oxalate, succinate, tartrate, malate, mandelate, trifluoroacetate, methane sulphonate and p-toluene sulphonate. X− is preferably an anion selected from chloride, bromide, iodide, sulphate, nitrate, acetate, maleate, oxalate, succinate or trifluoroacetate. More preferably X− is chloride, bromide, trifluoroacetate or methane sulphonate. Nonlimiting examples of pharmaceutically acceptable salts include but are not limited to glycolate, fumarate, mesylate, cinnamate, isethionate, sulfate, phosphate, diphosphate, nitrate, hydrobromide, hydroiodide, succinate, formate, acetate, dichloroacetate, lactate, p-toluenesulfonate, pamitate, pidolate. pamoate, salicylate, 4-aminosalicylate, benzoate, 4-acetamido benzoate, glutamate, aspartate, glycolate, adipate, alginate, ascorbate, besylate, camphorate, camphorsulfonate, camsylate, caprate, caproate, cyclamate, laurylsulfate, edisylate, gentisate, galactarate, gluceptate, gluconate, glucuronate, oxoglutarate, hippurate, lactobionate, malonate, maleate, mandalate, napsylate, napadisylate, oxalate, oleate, sebacate, stearate, succinate, thiocyanate, undecylenate, and xinafoate.

The term “effective amount” means an amount of a compound or composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, the route of administration, and like factors within the knowledge and expertise of the attending physician.

Compounds of the present invention may be combined with a pharmaceutically acceptable carrier to provide pharmaceutical compositions useful for treating the conditions or disorders. The particular carrier employed in the pharmaceutical compositions may vary depending upon the type of administration desired (e.g. intravenous, oral, topical, suppository, or parenteral). For example, in preparing the compositions in oral liquid dosage forms (e.g. suspensions, elixirs and solutions), typical pharmaceutical media include but not limited to water, glycols, oils, alcohols, flavoring agents, preservatives, coloring agents and the like. Similarly, for preparing oral solid dosage forms (e.g. powders, tablets, and capsules), carriers include but not limited to starches, sugars, diluents, granulating agents, lubricants, binders, disintegrating agents and the like.

Typical compositions include a compound of the invention and a pharmaceutically acceptable carrier. For example, the active compound will be mixed with a carrier, or diluted by a carrier, or enclosed within a carrier which can be in the form of an ampoule, capsule, sachet, paper, or other container. When the compound is mixed with a carrier, or when the carrier serves as a diluent, it can be solid, semi-solid, or liquid material that acts as a vehicle, excipient, or medium for the active compound. The compound can be adsorbed on a granular solid carrier, for example, contained in a sachet. Some examples of suitable carriers include but not limited to water, salt solutions, alcohols, polyethylene glycols. polyhydroxyethoxylated castor oil, peanut oil, olive oil, gelatin, lactose, terra alba, sucrose, dextrin. magnesium carbonate, sugar, cyclodextrin, amylose, magnesium stearate, talc, agar, pectin, acacia, stearic acid or lower alkyl ethers of cellulose, silicic acid, fatty acids, fatty acid amines, fatty acid mono glycerides and diglycerides, pentaerythritol fatty acid esters, polyoxyethylene. hydroxymethylcellulose, and polyvinylpyrrolidone. Similarly, the carrier or diluent can include any sustained release material known in the art such as glyceryl monostearate or glyceryl distearate, alone or mixed with a wax.

The term “one or more other pharmaceutical formulation(s)” refers to other active pharmaceutical ingredients or formulation that can work in combination with the pharmaceutical composition of the present disclosure. The other pharmaceutical formulations includes but not limited to Food and Drug Administration (FDA) approved drugs for the preliminary medication of AD patients and inflammation such as Aricept® (donepezil), Exelon® (rivastigmine), Razadyne® (galantamine), Namenda® (memantine), Nonsteroidal anti-inflammatory drugs (NSAIDs), Namzaric® (Donepezil and Memantine), Belsomra® (Suvorexant), and Aduhelm™ (Aducanumab).

In this specification, the prefix C_{x-y} as used in terms such as C_{x-y} alkyl and the like (where x and y are integers) indicates the numerical range of carbon atoms that are present in the group; for example, C₁₋₆ alkyl includes C₃ alkyl (propyl and isopropyl), C₄ alkyl (butyl, 1-methylpropyl, 2-methylpropyl, and t-butyl), and the like. Unless specifically stated, the bonding atom of a group may be any suitable atom of that group; for example, propyl includes prop-1-yl and prop-2-yl.

The term “C₁₋₆ alkyl” as used herein refers to a radical or group which may be saturated or unsaturated, linear or branched hydrocarbons, unsubstituted or mono- or poly-substituted.

The term “alkyl” refers to a mono-radical, branched or unbranched, saturated hydrocarbon chain having from 1 to 6 carbon atoms. This term is exemplified by groups such as n-butyl, iso-butyl, t-butyl, n-hexyl, and the like. The groups may be optionally substituted.

The term “alkenyl” refers to a mono-radical of a branched or unbranched unsaturated hydrocarbon group preferably having from 2, 3, 4, 5, to 6 carbon atoms and having 1, 2, 3, inter alia double bonds. The groups may be optionally substituted.

The term “alkynyl” refers to a mono-radical of a branched or unbranched unsaturated hydrocarbon group preferably having from 2, 3, 4, 5, to 6 carbon atoms and having 1, 2, 3, inter alia triple bonds. The groups may be optionally substituted.

The term “heteroalkyl” refers to an alkyl radical having 1 to 18 carbon atoms and one or more skeletal carbon atoms replaced by heteroatoms selected from oxygen, nitrogen and sulfur. The alkyl chain may be optionally substituted.

The term “heteroaryl” refers to an aromatic cyclic group having 5, 6, 7, 8, 9, 10, 11, 12, 13, 14, 15, 16, 17 or 18 carbon atoms and 1, 2, 3 or 4 heteroatoms selected from oxygen, nitrogen and sulfur within at least one ring. Such heteroaryl groups can have a single ring (e.g. pyridyl or furyl) or multiple condensed rings (e.g. indolizinyl, benzothiazolyl, or benzothienyl). Examples of heteroaryls include, but are not limited to, [1,2,4] oxadiazole, [1,3,4] oxadiazole, [1,2,4] thiadiazole, [1,3,4] thiadiazole, pyrrole, imidazole, pyrazole, pyridine, pyrazine, pyrimidine, pyridazine, indolizine, isoindole, indole, indazole, purine, quinolizine, isoquinoline, quinoline, phthalazine, quinoxaline, quinazoline, cinnoline, pteridine, carbazole, carboline, phenanthridine, acridine, phenanthroline, isothiazole, phenazine, isoxazole, phenoxazine, phenothiazine, furan, thiophene, oxazole, thiazole, triazole, triazine and the like.

The term “heterocyclyl” refers to a saturated or partially unsaturated group having a single ring or multiple condensed rings, having from 3 to 12 carbon atoms and from 1 to 10 hetero atoms, preferably 1, 2, 3 or 4 heteroatoms, selected from nitrogen, sulfur, phosphorus, and/or oxygen within the ring. Heterocyclic groups can have a single ring or multiple condensed rings, and include tetrahydrofuranyl, morpholinyl, piperidinyl, piperazinyl, dihydropyridinyl, tetrahydroquinolinyl and the like. The groups may be optionally substituted.

The term “hydroxyl” refers to an —OH moiety attached to a main chain of carbon atoms.

The term “aryl” refers to any mono- and poly-carbocyclic ring systems wherein the individual carbocyclic rings in the polyring systems are fused or attached to each other via a single bond and wherein at least one ring is aromatic. Unless otherwise indicated, substituents to the aryl ring systems can be attached to any ring atom, such that the attachment results in formation of a stable ring system.

The term “Aβ42 or Aβ” refers to amyloid-O peptide produced in the brain and is a 42 amino acid proteolytic product from the amyloid precursor protein. Amyloid peptide undergoes misfolding and aggregation to cause many pathological cascades and is considered biomarker for Alzheimer's disease. The peptide is considered as a biomarker for correlating with Alzheimer's disease (AD) onset, mild cognitive impairment, vascular dementia, and other cognitive disorders.

The term “tau” refers to a class of microtubule-associated protein (MAP), helping to maintain and stabilize the microtubule assembly in matured neurons. Tau interacts with tubulin and stimulates its assembly into microtubules to maintain the structure and function of neuronal cells. The self-aggregation of hyperphosphorylated Tau form intracellular neurofibrillary tangles and paired helical filaments that is associated with the onset of neurodegenerative disorders like AD and taupathies.

The term “α-syn” refers to alpha-synuclein, a small presynaptic protein that is encoded by the SNCA gene in humans. Alpha-synuclein regulates synaptic vesicle trafficking and subsequent neurotransmitter release. The misfolding of Alpha-synuclein into β-sheet secondary structure is mainly responsible for pathogenic α-Syn aggregation and LB (lewy body) formation, which further leads to a neurodegenerative disorder like Parkinson's disease. α-syn is also reported to play a role in AD.

The term “amylin” refers to islet amyloid polypeptide (IAPP), is a 37-residue peptide hormone and is co-secreted with insulin from the pancreatic β-cells. Amylin plays a role in glycemic regulation by slowing gastric emptying and promoting satiety, thereby preventing post-prandial spikes in blood glucose levels. The abnormal aggregation of amylin results in damage to pancreatic cells and leads to type 2 diabetes mellitus (T2DM).

The term “amyloid toxicity” refers to the toxic effect caused by amyloid aggregation.

The term “Nrf2” refers to nuclear factor erythroid 2-related factor 2, is a transcription factor of the leucine zipper family, and plays a central role in the induction of cytoprotective genes in response to oxidative stress. Nuclear factor erythroid 2-related factor 2 (Nrf2) is a transcription factor that gets translocated to the nucleus, induces the transcription of antioxidant genes in oxidative stress conditions. The translocation of Nrf2 to the nucleus is one of the marker events of oxidative stress and a decrease in oxidative stress reduces the translocation process.

The term “NF-kβ” refers to nuclear factor-kappa R is a protein complex that controls transcription of DNA, cytokine production and cell survival which regulates the inflammatory pathway, apoptosis, cell cycle, growth factors, synaptic modulation, and bone remodeling processes in bone-forming cells and bone resorption.

The term “EC50” refers to half maximal effective concentration which is the concentration of a substance that induces a desired response halfway between the baseline and maximum after a specified exposure time. It is commonly used as a measure of a drug's potency.

The term “NMI” refers to the naphthalene monoimide compounds and is denoted as NMIs or NMI compounds. NMIs denotes the compounds of Formula (I), its stereoisomers or a pharmaceutically acceptable salt thereof of the present disclosure. The compounds of the present disclosure are also referred to as “amyloid modulators” or “modulators” or “multifunctional modulators” as these compounds synergistically modulate various etiologies of AD. Amyloid modulators refer to NMI compounds of Formula (I), its stereoisomers or a pharmaceutically acceptable salt thereof, in the present disclosure which can modulate the aggregation of amyloid and modulate associated pathologies.

A term once described, the same meaning applies for it, throughout the disclosure.

The compound of Formula (I), and its polymorphs, stereoisomers, prodrugs, solvates, co-crystals, intermediates, pharmaceutically acceptable salts, and metabolites thereof can also be referred as “compounds of the present disclosure” or “compounds”.

Furthermore, the compound of Formula (I), can be its stereoisomer's, or pharmaceutically acceptable salts and compositions. It is understood that included in the family of compounds of Formula (I), are isomeric forms including diastereoisomers, enantiomers, tautomers, and geometrical isomers in “E” or “Z” configurational isomer or a mixture of E and Z isomers. It is also understood that some isomeric forms such as diastereomers, enantiomers and geometrical isomers can be separated by physical and/or chemical methods by those skilled in the art.

Compounds disclosed herein may exist as single stereoisomers, racemates and or mixtures of enantiomers and/or diastereomers. All such single stereoisomers, racemates and mixtures thereof are intended to be within the scope of the subject matter described.

Compounds disclosed herein include isotopes of hydrogen, carbon, oxygen, fluorine, chlorine, iodine and sulfur which can be incorporated into the compounds. Compounds of this disclosure wherein atoms were isotopically labeled can be used in metabolic studies, kinetic studies, and imaging techniques such as positron emission tomography used in understanding the tissue distribution of the drugs. Compounds of the disclosure where hydrogen is replaced with deuterium may improve the metabolic stability, and pharmacokinetics properties of the drug such as in vivo half-life.

As discussed in the background, there is still a dire need in the state of art for drug molecules essentially small molecules to combat neurodegenerative diseases or neuroinflammation disorders. There have been various reports on small molecules targeting dual pathological factors, as hybrid modulators, tripeptide analogues, peptidomimetics and natural products with multifunctional ability to target Aβ aggregation inhibition, metal-Aβ toxicity, ROS, oxidative stress, mitochondrial damage, and acetylcholine esterase (AChE). However, there is a need for the rational design of small molecules by integrating distinct bioactive pharmacophores to synergistically target multiple pathological factors of AD. Hence there require a specific strategy to design and synthesize NMI scaffolds and peptidomimetic modulators targeting AP/tau, autophagy, and multiple AD pathology.

The present disclosure provides a strategy to design multifunctional modulators by integrating multiple pharmacophore fragments to combat neurodegeneration. It has been observed that the misfolding and aggregation of Aβ peptides through β-sheet conformation is supported by hydrophobic interactions. The surface hydrophobicity is the major driving force for Aβ aggregation to form toxic oligomers and fibrils. The planar aromatic molecules with appropriate substitutions had been shown to effectively disrupt the amyloid aggregation through hydrophobic and hydrogen bonding interactions. The in vitro and in vivo evaluation of NMI compounds provided a potential candidate to effectively modulate amyloid burden and resulted in significant improvements in learning, memory, and cognitive functions. The major toxicity in AD is metal-Aβ complex formation, ROS generation and metal-dependent aggregation, wherein Cu (II) and Zn (II) play a critical role. Di-2-picolylamine (DPA) binds to metal ions and chelates with Zn (II), hence DPA moiety will act as a metal chelator on NMI scaffold. Further, it has been observed that the synthetic and natural products with catechol moiety exhibit metal chelation, antioxidant activity, reduce ROS levels, anti-neuroinflammatory property, and inhibited Aβ aggregation in vitro and in cellulo. Therefore, incorporation of DPA and dopamine is expected to enhance metal chelation, and exhibit antioxidant, and anti-neuroinflammatory properties. Thus, the compounds of present disclosure having NMIs with DPA and dopamine moieties effectively inhibit aggregation of Aβ and act as multifunctional modulators. Hence the present disclosure provides a multifunctional naphthalene monoimide compounds (NMIs) by incorporating bioactive pharmacophores to effectively modulate multiple aetiologies of AD. Accordingly the compounds of the present disclosure provides a synergistic effect of aggregation inhibition, metal chelation, ROS quenching, antioxidant, and anti-inflammatory activities. The modulation of Aβ, Aβ-metals and ROS triggered mitochondrial damage, neuroinflammation and synergistically rescue neuronal cells from multifaceted toxicity of AD.

Unless defined otherwise, all technical and scientific terms used herein have the same meaning as commonly understood by one of ordinary skill in the art to which this disclosure belongs. Although any methods and materials similar or equivalent to those disclosed herein can be used in the practice or testing of the disclosure, the preferred methods, and materials are now described. All publications mentioned herein are incorporated herein by reference.

The present disclosure is not to be limited in scope by the specific embodiments disclosed herein, which are intended for the purposes of exemplification only. Functionally-equivalent products, compositions, and methods are clearly within the scope of the disclosure, as disclosed herein.

In an embodiment of the present disclosure, there is provided a compound of Formula (I)

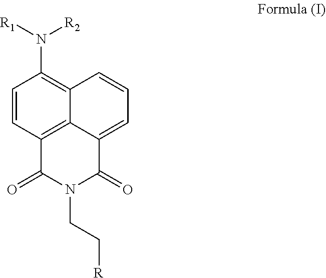

its stereoisomers or pharmaceutically acceptable salts thereof, wherein R is selected from C₆₋₁₀ aryl, C₅₋₁₈ heteroaryl, or NR₃R₄, wherein C₆₋₁₀ aryl is optionally substituted with two or more hydroxyl groups; R₁ and R₂ are independently selected from C₁₋₆ alkyl, C₂₋₆ alkenyl, or C₂₋₆ alkynyl, wherein C₁₋₆ alkyl is optionally substituted with one or more groups selected from C₆₋₁₀ aryl, C₁₋₁₈ heteroalkyl, C₅₋₁₈ heteroaryl, C₃₋₁₂ cycloalkyl, and C₃₋₁₂ heterocyclyl; and R₃ and R₄ are independently C₁₋₆ alkyl, wherein C₁₋₆ alkyl is optionally substituted with one or more groups selected from C₆₋₁₀ aryl, C₁₋₁₈ heteroalkyl, C₅₋₁₈ heteroaryl, C₃₋₁₂ cycloalkyl, and C₃₋₁₂ heterocyclyl.

In an embodiment of the present disclosure, there is provided a compound of Formula (I), its stereoisomers or pharmaceutically acceptable salts thereof as disclosed herein, wherein R is C₆₋₁₀ aryl substituted with two or more hydroxyl groups or NR₃R₄; R₁ and R₂ are independently selected from C₁₋₆ alkyl, C₂₋₆ alkenyl, or C₂₋₆ alkynyl, wherein C₁₋₆ alkyl is optionally substituted with one or more groups selected from C₆₋₁₀ aryl, C₁₋₁₈ heteroalkyl, C₅₋₁₈ heteroaryl, C₃₋₁₂ cycloalkyl, and C₃₋₁₂ heterocyclyl; and R₃ and R₄ are independently C₁₋₆ alkyl, wherein C₁₋₆ alkyl is optionally substituted with one or more groups selected from C₆₋₁₀ aryl, C₁₋₁₈ heteroalkyl, C₅₋₁₈ heteroaryl, C₃₋₁₂ cycloalkyl, and C₃₋₁₂ heterocyclyl.

In an embodiment of the present disclosure, there is provided a compound of Formula (I), its stereoisomers or a pharmaceutically acceptable salt thereof tas disclosed herein, wherein R is C₆₋₁₀ aryl substituted with two or more hydroxyl groups or NR₃R₄; R₁ and R₂ are independently selected from C₁₋₆ alkyl, wherein C₁₋₆ alkyl is optionally substituted with C₅₋₁₈ heteroaryl; and R₃ and R₄ are independently C₁₋₆ alkyl, wherein C₁₋₆ alkyl is optionally substituted with C₅₋₁₈ heteroaryl.

In an embodiment of the present disclosure, there is provided a compound of Formula (I), its stereoisomers or a pharmaceutically acceptable salt thereof as disclosed herein, wherein R is C₆₋₁₀ aryl substituted with two or more hydroxyl groups; R₁ and R₂ are independently C₁₋₆ alkyl, wherein C₁₋₆ alkyl is optionally substituted with C₅₋₁₈ heteroaryl.

In an embodiment of the present disclosure, there is provided a compound of Formula (I), its stereoisomers or a pharmaceutically acceptable salt thereof as disclosed herein, wherein R is NR₃R₄; R₁ and R₂ are independently C₁₋₆alkyl, wherein C₁₋₆ alkyl is optionally substituted with C₆₋₁₈ heteroaryl; and R₃ and R₄ are independently C₁₋₆ alkyl substituted with C₅₋₁₈ heteroaryl.

In an embodiment of the present disclosure, there is provided a compound of Formula (I), and its stereoisomers and pharmaceutically acceptable salts thereof as disclosed herein selected from a) 2-(3,4-dihydroxyphenethyl)-6-(dimethylamino)-1H-benzo[Ide]isoquinoline-1,3(2H)-dione; b) 2-(2-(bis(pyridin-2-ylmethyl)amino)ethyl)-6-(dimethylamino)-1H-benzo[Ide]isoquinoline-1,3(2H)-dione; c) 6-(bis(pyridin-2-ylmethyl)amino)-2-(3,4-dihydroxyphenethyl)-1H-benzo[Ide]isoquinoline-1,3(2H)-dione; and d) 6-(bis(pyridin-2-ylmethyl)amino)-2-(2-(bis(pyridin-2-ylmethyl)amino)ethyl)-1H-benzo[de]isoquinoline-1,3(2H)-dione.

In an embodiment of the present disclosure, there is provided a process of preparation of compound of Formula (I), its stereoisomers or a pharmaceutically acceptable salt thereof as disclosed herein, and its stereoisomers and pharmaceutically acceptable salts thereof, the process comprising: a) reacting a compound of Formula II with Formula III in the presence of a base and a solvent, to obtain a compound of Formula (I)

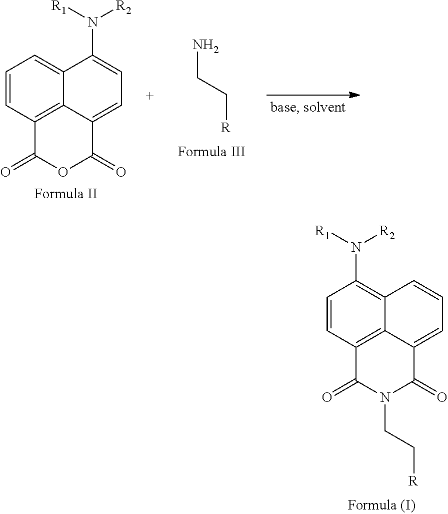

wherein R is selected from C₆₋₁₀ aryl, C₅₋₁₈ heteroaryl, or NR₃R₄, wherein C₆₋₁₀ aryl is optionally substituted with two or more hydroxyl groups; R₁ and R₂ are independently selected from C₁₋₆ alkyl, C₂₋₆ alkenyl, or C₂₋₆ alkynyl, wherein C₁₋₆ alkyl is optionally substituted with one or more groups selected from C₆₋₁₀ aryl, C₁₋₁₈ heteroalkyl, C₅₋₁₈ heteroaryl, C₃₋₁₂ cycloalkyl, and C₃₋₁₂ heterocyclyl; and R₃ and R₄ are independently C₁₋₆ alkyl, wherein C₁₋₆ alkyl is optionally substituted with one or more groups selected from C₆₋₁₀ aryl, C₁₋₁₈ heteroalkyl, C₅₋₁₈ heteroaryl, C₃₋₁₂ cycloalkyl, and C₃₋₁₂ heterocyclyl.

In an embodiment of the present disclosure, there is provided a process of preparation of compound of Formula (I), its stereoisomers or a pharmaceutically acceptable salt thereof as disclosed herein, wherein the base is selected from organic or inorganic bases including 1,8-diazabicyclo(5.4.0)undec-7-ene (DBU), N,N-diisopropylethylamine (DIPEA), triethylamine (Et₃N), C₁₋₁₀ alkyl amine, pyridine, or combinations thereof; and the solvent is selected from dimethyl formamide, ethanol, methanol, isopropyl alcohol, dimethylsulphoxide (DMSO), benzene, toluene, xylene, or combinations thereof. In another embodiment of the present disclosure, the base is triethyl amine and the solvent is ethanol.

In an embodiment of the present disclosure, there is provided a process of preparation of compound of Formula (I), its stereoisomers or a pharmaceutically acceptable salt thereof as disclosed herein, wherein reacting the compound of Formula II with Formula III is carried out at a temperature in the range of 80 to 110° C. for a time period in the range of 2 to 5 hours. In another embodiment of the present disclosure, reacting the compound of Formula II with Formula III is carried out at a temperature of 90° C. for a time period in the range of 2 to 4 hours.

In an embodiment of the present disclosure, there is provided a compound of Formula (I), its stereoisomers or a pharmaceutically acceptable salt thereof as disclosed herein, wherein the compounds of Formula (I), its stereoisomers or a pharmaceutically acceptable salt thereof provides reversal of cognitive decline or improvement of cognitive decline.

In an embodiment of the present disclosure, there is a pharmaceutical composition comprising the compound of Formula (I), its stereoisomers or a pharmaceutically acceptable salt thereof, with a pharmaceutically acceptable carrier, optionally in combination with one or more other pharmaceutical formulations.

In an embodiment of the present disclosure, there is a pharmaceutical composition comprising the compound of Formula (I), its stereoisomers or a pharmaceutically acceptable salt thereof, wherein the composition is in a form selected from tablet, capsule, powder, syrup, solution, aerosol, or suspension.

In an embodiment of the present disclosure, there is provided a compound of Formula (I), its stereoisomers or a pharmaceutically acceptable salt thereof, for use in the manufacture of a medicament for treating a neurodegenerative disease or a neuroinflammation disorder.

In an embodiment of the present disclosure, there is provided a compound of Formula (I), its stereoisomers or a pharmaceutically acceptable salt thereof, wherein the disease or disorder is selected from Alzheimer's disease (AD), Parkinson's disease (PD), prion diseases, polyglutamine expansion diseases, Huntington's disease (HD), tauopathies, frontotemporal dementia associated with tau-immunoreactive inclusions (FTD-tau), progressive supranuclear palsy (PSP), corticobasal degeneration (CBD), amyotrophic lateral sclerosis (ALS), lewibody disease, or spinal muscular atrophy.

In an embodiment of the present disclosure, there is provided a compound of Formula (I), its stereoisomers or a pharmaceutically acceptable salt thereof, wherein the compounds of Formula (I) modulate aggregation of one or more protein selected from a group consisting of Aβ, tau, α-syn, polyglutamine and amylin (IAPP).

In an embodiment of the present disclosure, there is provided a compound of Formula (I), its stereoisomers or a pharmaceutically acceptable salt thereof, wherein the compounds of Formula (I) synergistically modulate metal independent and dependent amyloid toxicity, scavenge reactive oxidative species (ROS), alleviate oxidative stress, emulate Nrf2 mediated stress response, mitochondrial damage, microglial activation and neuroinflammation.

In an embodiment of the present disclosure, there is provided a compound of Formula (I), its stereoisomers or a pharmaceutically acceptable salt thereof, wherein the compounds of Formula (I) suppresses NF-kβ.

In an embodiment of the present disclosure, there is a pharmaceutical composition comprising the compound of Formula (I), its stereoisomers or a pharmaceutically acceptable salt thereof as disclosed herein, wherein the pharmaceutical composition modulate aggregation of one or more protein selected from a group consisting of Aβ, tau, α-syn, polyglutamine and amylin (IAPP).

In an embodiment of the present disclosure, there is a pharmaceutical composition comprising the compound of Formula (I), its stereoisomers or a pharmaceutically acceptable salt thereof as disclosed herein, wherein the pharmaceutical composition provides reversal of cognitive decline or improvement of cognitive decline.

In an embodiment of the present disclosure, there is a pharmaceutical composition comprising the compound of Formula (I), its stereoisomers or a pharmaceutically acceptable salt thereof as disclosed herein, for use in the manufacture of a medicament for treating a disease or disorder selected from Alzheimer's disease (AD), Parkinson's disease (PD), prion diseases, polyglutamine expansion diseases, Huntington's disease (HD), tauopathies, frontotemporal dementia associated with tau-immunoreactive inclusions (FTD-tau), progressive supranuclear palsy (PSP), corticobasal degeneration (CBD), amyotrophic lateral sclerosis (ALS), lewibody disease, and spinal muscular atrophy.

In an embodiment of the present disclosure, there is provided a compound of Formula (I), its stereoisomers or a pharmaceutically acceptable salt thereof as disclosed herein for the treatment of a condition mediated by a neurodegenerative disease or neuroinflammation disorder, wherein an effective amount of the compound is administered to a subject in need thereof.

In an embodiment of the present disclosure, there is provided a pharmaceutical composition comprising the compound of Formula (I), its stereoisomers or a pharmaceutically acceptable salt thereof as disclosed herein for the treatment of a condition mediated by a neurodegenerative disease or neuroinflammation disorder, wherein an effective amount of the composition is administered to a subject in need thereof.

In an embodiment of the present disclosure, there is provided a compound of Formula (I), its stereoisomers or a pharmaceutically acceptable salt thereof as disclosed herein for the treatment of a condition mediated by a neurodegenerative disease or a neuroinflammation disorder by administering a combination of the compound of Formula (I) with other clinically relevant immune modulator agents to a subject in need of thereof.

In an embodiment of the present disclosure, there is provided a pharmaceutical composition comprising the compound of Formula (I), its stereoisomers or a pharmaceutically acceptable salt thereof as disclosed herein for the treatment of a condition mediated by a neurodegenerative disease or a neuroinflammation disorder by administering a combination of the composition with other clinically relevant immune modulator agents to a subject in need of thereof.

In an embodiment of the present disclosure, there is provided a method for the treatment of a condition mediated by a neurodegenerative disease or a neuroinflammation disorder, said method comprising administering to a subject an effective amount of the compound of Formula (I), its stereoisomers or a pharmaceutically acceptable salt thereof as disclosed herein.

In an embodiment of the present disclosure, there is provided a method for the treatment of a condition mediated by a neurodegenerative disease or a neuroinflammation disorder, said method comprising administering to a subject an effective amount of the pharmaceutical composition as disclosed herein.

In an embodiment of the present disclosure, there is provided a method of treatment of a condition mediated by a neurodegenerative disease or a neuroinflammation disorder, said method comprising administering a combination of the compound of Formula (I), its stereoisomers or a pharmaceutically acceptable salt thereof as disclosed herein with other clinically relevant immune modulator agents to a subject in need of thereof.

In an embodiment of the present disclosure, there is provided a method of treatment of a condition mediated by a neurodegenerative disease or a neuroinflammation disorder, said method comprising administering a combination of the pharmaceutical composition as disclosed herein, with other clinically relevant immune modulator agents to a subject in need of thereof.

In an embodiment of the present disclosure, there is provided a method for the treatment of a condition mediated by the disease or disorder selected from Alzheimer's disease (AD), Parkinson's disease (PD), prion diseases, polyglutamine expansion diseases, Huntington's disease (HD), tauopathies, frontotemporal dementia associated with tau-immunoreactive inclusions (FTD-tau), progressive supranuclear palsy (PSP), corticobasal degeneration (CBD), amyotrophic lateral sclerosis (ALS), lewibody disease, and spinal muscular atrophy.

In an embodiment of the present disclosure, there is provided a method for the treatment of a condition mediated by a neurodegenerative disease or a neuroinflammation disorder, wherein the compounds of Formula (I), its stereoisomers or a pharmaceutically acceptable salt thereof, synergistically modulate metal independent and dependent amyloid toxicity, scavenge ROS, alleviate oxidative stress, emulate Nrf2 mediated stress response, mitochondrial damage, microglial activation and neuroinflammation.

In an embodiment of the present disclosure, there is provided a use of the compound of Formula (I), its stereoisomers or a pharmaceutically acceptable salt thereof as disclosed herein for treatment of a condition mediated by aggregation of one or more protein selected from Aβ42, tau, α-syn, polyglutamine and amylin (IAPP).

In an embodiment of the present disclosure, there is provided a use of the pharmaceutical composition as disclosed herein for the treatment of a condition mediated by aggregation of one or more protein selected from Aβ42, tau, α-syn, polyglutamine and amylin (IAPP).

In an embodiment of the present disclosure, there is provided a use of the compound of Formula (I), its stereoisomers or a pharmaceutically acceptable salt thereof as disclosed herein with other clinically relevant agents or biological agents for the treatment of a condition mediated by aggregation of one or more protein selected from Aβ42, tau, α-syn, polyglutamine and amylin (IAPP).

In an embodiment of the present disclosure, there is provided a use of the compound of the pharmaceutical composition as disclosed herein with other clinically relevant agents or biological agents for the treatment of a condition mediated by aggregation of one or more protein selected from Aβ42, tau, α-syn, polyglutamine and amylin (IAPP).

In an embodiment of the present disclosure, there is provided a use of the Formula (I), its stereoisomers or a pharmaceutically acceptable salt thereof as disclosed herein, wherein the compounds of Formula (I) synergistically modulate metal independent and dependent amyloid toxicity, scavenge ROS, alleviate oxidative stress, emulate Nrf2 mediated stress response, mitochondrial damage, microglial activation and neuroinflammation.

In an embodiment of the present disclosure, there is provided a use of the compound of Formula (I), its stereoisomers or a pharmaceutically acceptable salt thereof as disclosed herein with other clinically relevant agents or biological agents, wherein the compounds of Formula (I) synergistically modulate metal independent and dependent amyloid toxicity, scavenge ROS, alleviate oxidative stress, emulate Nrf2 mediated stress response, mitochondrial damage, microglial activation and neuroinflammation.

In an embodiment of the present disclosure, there is provided a use of the Formula (I), its stereoisomers or a pharmaceutically acceptable salt thereof as disclosed herein, wherein the compounds of Formula (I), its stereoisomers or a pharmaceutically acceptable salt thereof suppresses NF-kβ.

In an embodiment of the present disclosure, there is provided a use of the compound of Formula (I), its stereoisomers or a pharmaceutically acceptable salt thereof as disclosed herein with other clinically relevant agents or biological agents, wherein the compounds of Formula (I), its stereoisomers or a pharmaceutically acceptable salt thereof suppresses NF-kβ.

In an embodiment of the present disclosure, there is provided a use of the pharmaceutical composition as disclosed herein, wherein the composition synergistically modulates metal independent and dependent amyloid toxicity, scavenge ROS, alleviate oxidative stress, emulate Nrf2 mediated stress response, mitochondrial damage, microglial activation and neuroinflammation.

In an embodiment of the present disclosure, there is provided a use of the pharmaceutical composition with other clinically relevant agents or biological agents, wherein the composition synergistically modulates metal independent and dependent amyloid toxicity, scavenge ROS, alleviate oxidative stress, emulate Nrf2 mediated stress response, mitochondrial damage, microglial activation and neuroinflammation.

In an embodiment of the present disclosure, there is provided a use of the pharmaceutical composition as disclosed herein, wherein the composition suppresses NF-kβ.

In an embodiment of the present disclosure, there is provided a use of the pharmaceutical composition with other clinically relevant agents or biological agents, wherein the composition suppresses NF-kβ.

In an embodiment of the present disclosure, there is provided a compound of Formula (I), its stereoisomers or a pharmaceutically acceptable salt thereof as disclosed herein for use in treatment of a condition mediated by aggregation of one or more protein selected from Aβ42, tau, α-syn, polyglutamine and amylin (IAPP).

In an embodiment of the present disclosure, there is provided a pharmaceutical composition comprising the compound of Formula (I), its stereoisomers or a pharmaceutically acceptable salt thereof as disclosed herein for use in treatment of a condition mediated by aggregation of one or more protein selected from Aβ42, tau, α-syn, polyglutamine and amylin (IAPP).

In an embodiment of the present disclosure, there is provided a compound of Formula (I), its stereoisomers or a pharmaceutically acceptable salt thereof as disclosed herein with other clinically relevant agents or biological agents for treatment of a condition mediated by aggregation of one or more protein selected from Aβ42, tau, α-syn, polyglutamine and amylin (IAPP).

In an embodiment of the present disclosure, there is provided a pharmaceutical composition comprising the compound of Formula (I), its stereoisomers or a pharmaceutically acceptable salt thereof as disclosed herein with other clinically relevant agents or biological agents for treatment of a condition mediated by aggregation of one or more protein selected from Aβ42, tau, α-syn, polyglutamine and amylin (IAPP).

Although the subject matter has been described in considerable detail with reference to certain embodiments thereof, other embodiments are possible. As such, the spirit and scope of the disclosure should not be limited to the description of the embodiments contained herein.

### EXAMPLES

The disclosure will now be illustrated with the working examples, which is intended to illustrate the working of disclosure and not intended to take restrictively to imply any limitations on the scope of the present disclosure. Unless defined otherwise, all technical and scientific terms used herein have the same meaning as commonly understood to one ordinary person skilled in the art to which this disclosure belongs. Although methods and materials similar or equivalent to those disclosed herein can be used in the practice of the disclosed methods and compositions, the exemplary methods, devices and materials are disclosed herein. It is to be understood that this disclosure is not limited to particular methods, and experimental conditions described, as such methods and conditions may apply.

### Materials and Methods

All the chemicals and solvents were procured from Merck or spectrochem and used as received unless mentioned. TLC plates (Silica gel 60 F254) were purchased from Merck and used for reaction monitoring and visualized by UV light (254 nm and 365 nm) and ninhydrin. Column chromatography was performed using 100-200 mesh silica gel as stationary phase. Aβ peptide was purchased from Invitrogen and used without further purification. The acrylamide, bis-acrylamide, ammonium persulfate (APS), tetramethyl ethylenediamine (TEMED), 2,2-diphenyl-1-picrylhydrazyl (DPPH), 2,2′-azino-bis(3-ethylbenzothiazoline-6-sulfonic acid (ABTS), 3-(4,5-dimethylthiazol-2-yl)- 2,5-diphenyl tetrazolium bromide (MMT), metal salts and E. coli lipo polysachharide (LPS) reagents were purchased from Merck. Polyvinylidene difluoride (PVDF) membrane, Broadford reagent, SDS-PAGE sample loading buffer, protein ladder and enhanced chemiluminescence (ECL) reagent were purchased from BioRad. Mica discs were purchased from Ted Pella and used without any modifications. The cell media DMEM-F12 (Dulbecco's Modified Eagle Medium), fetal bovine serum (FBS), horse serum (HS), goat serum (GS), penicillin-streptomycin (PS), and Dulbecco's phosphate buffer saline (dPBS) were purchased from Gibco. Rhodamine phalloidin, 4′,6-diamidino-2-phenylindole (DAPI), MitoOrange and MitoSox were procured from Invitrogen. Mito-TG is an in-house mitochondrial probe, synthesis and use is reported elsewhere. Radioimmunoprecipitation assay (RIPA) lysis buffer was purchased from G-Bioscience and protease inhibitor cocktail (50×) from Promega. Sterile T25 flasks (Eppendorf), 96 well plate (Thermos Fischer Scientific) and confocal dishes (SPL lifesciences) were purchased and used without further sterilization. 1H NMR were recorded on Bruker AV-400 (400 MHz) spectrometer (TMS: tetramethyl silane was used as internal standard). ¹H NMR chemical shifts were reported in ppm downfield from TMS. Splitting patterns are abbreviated as s: singlet, d: doublet, dd: doublet of doublet, t: triplet, q: quartet, dq: doublet of quartet, p: pentet, m: multiplet. ¹³C NMR spectra were recorded on Bruker AV-400 (101 MHz) and Jeol-600 (150 MHz) spectrophotometers. High-resolution mass spectra (HRMS) were obtained from Agilent 6538 UHD HRMS/Q-TOF high-resolution spectrometer. The absorption and fluorescence spectra were recorded on Agilent Cary Eclipse Fluorescence Spectrophotometer and Cary Series UV-Vis-NIR spectrophotometer, respectively. The blots were imaged in ChemiDoc (BioRad) gel documentation instrument and analyzed by ImageJ. For 96 well plates, absorbance and fluorescence measurement were performed in Spectramax i3 (Molecular devices) plate reader instrument. Live cell fluorescence imaging was carried out in Leica DMi8 fluorescence microscope equipped with live cell set up. Images were processed using Huygens and analysed by ImageJ. The confocal imaging was performed using Olympus Fluoview-3000 confocal laser scanning microscope and analysed by ImageJ software. Biological atomic force microscopy (Bio-AFM) imaging was carried out in Bruker Bioscope Resolve AFM integrated with Olympus Fluoview-3000 Confocal laser scanning microscope. Table 1 depicts the list of antibodies used in the present disclosure.

TABLE 1

| SI | | | | | |
|---|---|---|---|---|---|
| No | Antibody | Application | Dilution | Origin | Vendor |
| 1 | OC | Dot blot and | 1:1000 | Rabbit | Merk Milipore |
| | | western blot | | | |
| 2 | A11 | Dot blot | 1:1000 | Rabbit | Invitrogen |
| 3 | Nrf2 | Immunoflu- | 1:500 | Rabbit | Invitrogen |
| | | orescence | | | |
| 4 | Cyt c | Western Blot | 1:2000 | Rabbit | Invitrogen |
| 5 | Iba1 | Immunoflu- | 1:200 | Rabbit | Cell signaling |
| | | orescence | | | technology |
| 6 | NF-kβ | Western Blot | 1:1000 | Rabbit | Cell signaling |
| | | | | | technology |
| 7 | TNFα | Western Blot | 1:1000 | Rabbit | Cell signaling |
| | | | | | technology |
| 8 | IL6 | Western Blot | 1:1000 | Rabbit | Cell signaling |
| | | | | | technology |
| 9 | βActin | Western Blot | 1:1000 | Rabbit | Invitrogen |
| 10 | GAPDH | Western Blot | 1:1000 | Rabbit | Invitrogen |
| 11 | HRP-conjugated | Dot blot and | 1:5000 | Goat | Invitrogen |
| | secondary | Western Blot | | | |
| 12 | Alexa488- | Immunoflu- | 1:1000 | Goat | Invitrogen |
| | conjugated | orescence | | | |
| | secondary | | | | |

### Example 1

### Synthesis of Compounds of Formula (I)

The compounds of Formula (I) (NMIs) were synthesized wherein R is C₆ aryl substituted with two hydroxyl groups or NR₃R₄ with R₃ and R₄ are independently C₁ alkyl, wherein C₁ alkyl is optionally substituted two C₅ heteroaryl; and R₁ and R₂ is independently C₁ alkyl, and wherein C₁ alkyl is optionally substituted with C₅ heteroaryl. Pharmaceutically acceptable salts of the compounds may be obtained as per procedures reported in the literature. Scheme 1 represents the general scheme of preparation of compounds of the present disclosure. Scheme 2 represents synthetic preparation of different compounds from the parent naphthalene monoanhydride molecule.

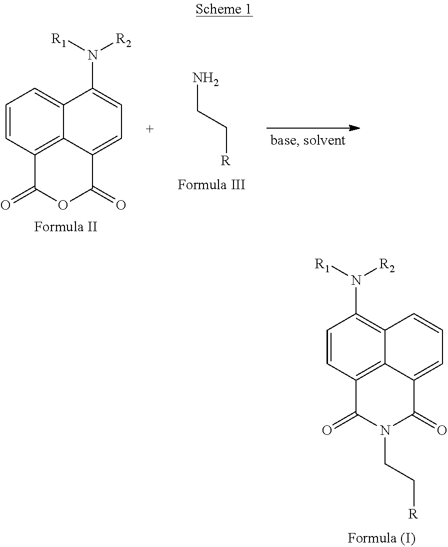

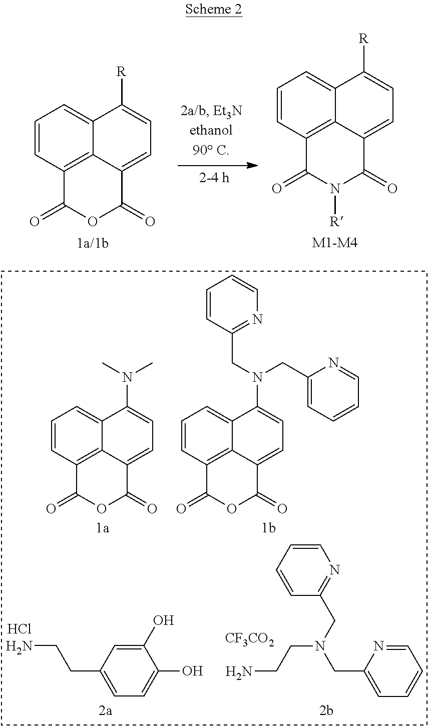

The compounds of the present disclosure were prepared by scheme 2 as depicted above. N,N′-dimethyl naphthalene monoanhydride (1a) or N,N′-di(2-picolylamine) naphthalene monoanhydride (1b) (80 mg, 0.33 mmol) was mixed with dopamine hydrochloride (2a) or N′,N′-bis(pyridin-2-ylmethyl)ethane-1,2-diamine (2b) (94 mg, 0.50 mmol) in ethanol as solvent and triethylamine as a base under reflux conditions for 2 h and the reaction mixture was allowed to reflux for 2 h. The reaction was monitored using thin layered chromatography (TLC). After completion, the reaction mixture was brought to room temperature and volatiles were removed under reduced pressure to obtain the crude product. The crude product was dissolved in water and extracted with ethyl acetate/dichloromethane. The combined organic layers were dried over anhydrous sodium sulphate and evaporated to dryness under reduced pressure. The obtained product was purified by silica gel column chromatography using dichloromethane/methanol as mobile phase. The products obtained (M1 to M4) were ascertained by ¹H and ¹³C NMR spectroscopy, high performance liquid chromatography (HPLC) and high-resolution mass spectrometry (HR-MS) analyses.

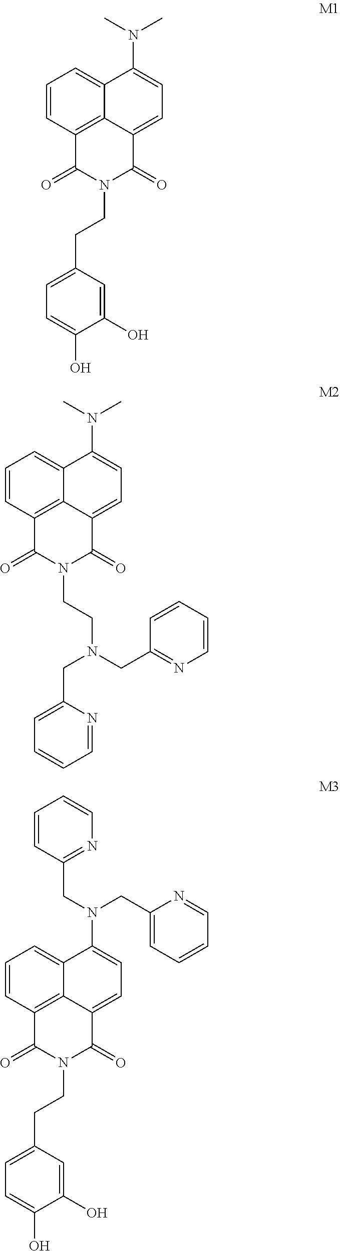

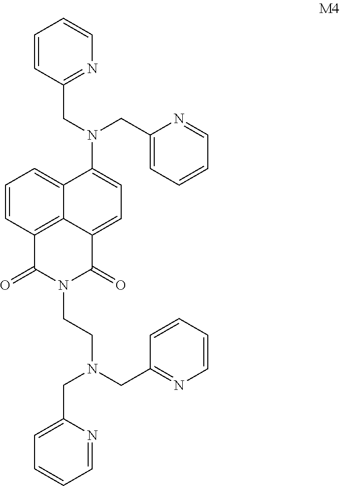 [SMILES: O=C1c2cccc3c(N(Cc4ccccn4)Cc4ccccn4)ccc(c23)C(=O)N1CCN(Cc1ccccn1)Cc1ccccn1]

### 2-(3,4-dihydroxyphenethyl)-6-(dimethylamino)-1H-benzo[de]isoquinoline-1,3(2H)-dione (M1)

Yellow solid (100 mg, 81%). ¹H NMR (400 MHz, DMSO-D₆) δ 8.81 (s, 1H), 8.65 (s, 1H), 8.49 (d, J=8.5 Hz, 1H), 8.43 (d, J=7.2 Hz, 1H), 8.32 (d, J=8.3 Hz, 1H), 7.73 (t, J=7.9 Hz, 1H), 7.19 (d, J=8.3 Hz, 1H), 6.68 (d, J=1.6 Hz, 1H), 6.64 (d, J=7.9 Hz, 1H), 6.49 (dd, J=8.0, 1.6 Hz, 1H), 4.20-4.08 (m, 2H), 3.08 (s, 6H), 2.75-2.64 (m, 2H). ¹³C NMR (100 MHz, DMSO-D₆) δ 163.5, 162.8, 156.5, 145.1, 143.7, 132.2, 131.5, 130.5, 129.6, 125.0, 124.2, 122.3, 119.2, 115.9, 115.6, 113.3, 113.0, 44.4, 41.1, 33.0. HRMS (ESI-TOF) m/z: Found: 377.1493 [M+H]+, Calcd. for C₂₂H₂₀N₂O₄ 377.1501.

### 2-(2-(bis(pyridin-2-ylmethyl)amino)ethyl)-6-(dimethylamino)-1H-benzo[de]isoquinoline-1,3(2H)-dione (M2)

Yellow solid (67 mg, 73%). ¹H NMR (400 MHz, CDCl₃) δ 8.47 (dd, J=15.0, 7.9 Hz, 2H), 8.41 (d, J=8.3 Hz, 3H), 7.66 (t, J=7.9 Hz, 1H), 7.39 (d, J=7.7 Hz, 2H), 7.30 (t, J=7.6 Hz, 2H), 7.12 (d, J=8.2 Hz, 1H), 7.04-6.94 (m, 2H), 4.40 (t, J=6.3 Hz, 2H), 3.93 (s, 4H), 3.11 (s, 6H), 2.93 (t, J=6.2 Hz, 2H). ¹³C NMR (100 MHz, DMSO-D₆) δ 164.5, 164.0, 159.7, 157.0, 148.8, 136.3, 132.7, 131.2, 131.1, 130.4, 125.4, 125.0, 123.3, 123.0, 121.9, 115.2, 113.5, 60.4, 52.1, 44.9, 37.9. HRMS (ESI-TOF) m/z: Found: 466.2241 [M+H]+, Calcd. for C₂₈H₂₈N₅O₂ 466.2243.

### 6-(bis(pyridin-2-ylmethyl)amino)-2-(3,4-dihydroxyphenethyl)-1H-benzo[de]isoquinoline-1,3(2H)-dione (M3)

Bright yellow solid (110 mg, 83%). ¹H NMR (400 MHz, DMSO-D₆) δ 8.96 (d, J=8.5 Hz, 1H), 8.80 (s, 1H), 8.64 (s, 1H), 8.54 (d, J=4.3 Hz, 2H), 8.49 (d, J=7.2 Hz, 1H), 8.25 (d, J=8.2 Hz, 1H), 7.82 (t, J=7.9 Hz, 1H), 7.73 (t, J=7.7 Hz, 2H), 7.46 (d, J=7.7 Hz, 2H), 7.27 (dd, J=10.1, 5.0 Hz, 3H), 6.67 (s, 1H), 6.63 (d, J=7.9 Hz, 1H), 6.49 (d, J=8.0 Hz, 1H), 4.73 (s, 4H), 4.20-4.07 (m, 2H), 2.74-2.65 (m, 2H). ¹³C NMR (100 MHz, DMSO-D₆) δ 163.4, 162.8, 157.2, 153.8, 149.2, 145.1, 143.7, 136.8, 131.5, 130.6, 129.5, 129.4, 125.9, 122.6, 122.5, 122.3, 119.2, 117.1, 115.9, 115.6, 115.1, 59.1, 41.2, 33.0. HRMS (ESI-TOF) m/z: Found: 531.2029 [M+H]+, Calcd. for C₃₂H₂₇N₄O₂ 531.2032.

### 6-(bis(pyridin-2-ylmethyl)amino)-2-(2-(bis(pyridin-2-ylmethyl)amino)ethyl)-1H-benzo[de]isoquinoline-1,3(2H)-dione (M4)

Yellow solid (140 mg, 57%). ¹H NMR (400 MHz, CDCl₃) δ 8.92 (dd, J=8.5, 1.0 Hz, 1H), 8.60-8.55 (m, 2H), 8.52 (dd, J=7.3, 1.0 Hz, 1H), 8.37-8.32 (m, 2H), 8.26 (d, J=8.1 Hz, 1H), 7.72 (dd, J=8.4, 7.3 Hz, 1H), 7.60 (td, J=7.7, 1.8 Hz, 2H), 7.40 (dd, J=13.4, 7.8 Hz, 4H), 7.31 (td, J=7.7, 1.6 Hz, 2H), 7.21 (d, J=8.2 Hz, 1H), 7.17 (ddd, J=7.5, 4.9, 0.9 Hz, 2H), 7.01-6.93 (m, 2H), 4.76 (s, 4H), 4.38 (t, J=6.2 Hz, 2H), 4.00 (d, J=11.1 Hz, 4H), 2.98 (t, J=6.2 Hz, 2H). ¹³C NMR (150 MHz, DMSO-D₆) δ 164.4, 163.8, 157.4, 153.8, 149.7, 148.4, 136.9, 132.1, 131.2, 130.5, 130.2, 126.8, 126.1, 123.5, 123.4, 122.7, 122.5, 122.2, 117.7, 116.6, 59.9, 59.8, 51.9, 37.6. HRMS (ESI-TOF) m/z: Found: 620.2750 [M+H]+, Calcd. for C₃₈H₃₄N₇O₂ 620.2774.

### Example 2

### Experimental Details

### Dot Blot Assay

Aggregation modulation by toxic Aβ aggregates were assessed and were quantified by dot blot assay. Aβ peptide (10 μM) was dissolved in phosphate buffered saline (PBS, 10 mM, pH 7.4), incubated alone and with NMIs (20 μM) at 37° C. for 48 h. For concentration dependent inhibition, 10 μM Aβ peptide was incubated with 1, 2, 5, 10 and 20 μM of NMIs and incubated. Samples for oligomer inhibition studies were prepared by incubating Aβ alone and with NMIs at 4° C. for 24 and subjected to dot blot analysis. For dissolution studies, Aβ peptide alone was incubated in PBS for 48 h to prepare fibrils and then incubated with NMIs further for 48 h. For dot blot experiment, 5 μL of sample was blotted on activated PVDF (polyvinylidene fluoride) membrane in triplicates and air dried. The membrane was blocked with skimmed milk powder for 1 h at room temperature. The blot paper was washed with tris-buffered saline with 0.01% Tween 20 (TBST) three times for 5 min. Blot paper was treated with fibril specific OC primary antibody (Rabbit, 1:1000 dilution) overnight at 4° C. For oligomers blot A11 anti-oligomer antibody (Rabbit 1:1000) was used. Blot was washed thrice with TBST and treated with HRP-conjugated antirabbit secondary antibody (1:5000 dilutions) for 1 h at room temperature. The bolt was washed thrice for 5 min with TBST and developed with ECL reagent (Biorad) and imaged with ChemiDoc BioRad gel documentation system in autoexposure mode. Blots were analyzed and quantified by ImageJ software.

### Native Polyacrylamide Gel Electrophoresis and Western Blot Analysis

The samples were prepared by incubating Aβ42 with specific concentration of NMI compounds of present disclosure and metal ions for 48 h and then subjected to 6% native polyacrylamide gel electrophoresis (Native-PAGE) in tris-glycine buffer. The lower molecular weight aggregates permeabilized through the gel and resolved in the gel. Higher molecular aggregates stuck in the upper part of wells and potentially carried in gel. Then the protein was transferred to PVDF membrane for 4 h at cold condition maintained using ice cold transfer buffer and immersing the transfer apparatus in ice. Then the blots were continued similar to dot blots.

### Atomic Force Microscopy (AFM) Imaging

AFM imaging was done to understand the morphological characteristics of Aβ and NMI treated samples. The samples from Aβ aggregation inhibition and dissolution studies were used for AFM imaging. 25 μL of sample was drop-casted on freshly cleaved mica substrate. The Aβ samples were allowed to adhere to substrate surface for 15 min, washed with filtered MiliQ water and air dried. The samples were imaged in Scan assist Air mode in Bioscope resolve AFM system (Bruker). The images were processed and analyzed using NanoScope analysis software.

### Antioxidant Assays

### DPPH Radical Scavenging Assay

For performing DPPH (2,2-diphenyl-1-picrylhydrazyl) radical scavenging assay, different concentration of NMIs were prepared in methanol and divided 100 μL to 96 well plates in 4 replicates. Equal volume of DPPH (200 μM) solution prepared in methanol was added and incubated at 37° C. for 30 min in dark condition. Absorbance at 517 nm wavelength was measured to calculate the amount of radicals quenched relative to control. Ascorbate, a natural antioxidant was taken as positive control for comparison of antioxidant capacity. The percentage radical quenching was calculated using the formula below. Percentage radical scavenging=[(Abs−Abs C)/Abs]×100 Where Abs is absorbance of sample, and Abs C is absorbance of control.

### ABTS Radical Scavenging Assay

ABTS (2, 2′-azino-bis(3-ethylbenzothiazoline-6-sulfonic acid)) in presence of potassium persulfate generated stable nitrogen radical which strongly absorbs at 734 nm. To generate ABTS radical, equal volume of 7 mM of ABTS in water was mixed with 2.45 mM of potassium per sulfate and incubated in dark for 14 h at room temperature. The radical solution was diluted to 200 μM in methanol to obtain working solution. Then 100 μL of different concentration of NMIs were mixed with 100 μL of ABTS solution in 96 well plate and incubated for 30 min in dark at room temperature. The absorbance at 734 nm was recorded and calculated the percentage radical quenching similar to DPPH assay.

### General Cell Culture

The SH-SY5Y cells were cultured in DMEM-F12 (Dulbecco's Modified Eagle Medium) media with 10% FBS (fetal bovine serum) and 1% PS (penicillin-streptomycin) and incubated in humidified incubator with 5% CO₂ at 37° C. temperature. The cells were cultured in T25 flask trypsinised at 80% confluency and seeded further as per the requirement. C6 microglial cells were cultured in DMEMF-12 media with 10% HS (horse serum) and 1% PS and incubated in humidified incubator with 5% CO₂ at 37° C. temperature.

### MTT (3-(4,5-dimethylthiazol-2-yl)-2,5-diphenyltetrazolium bromide) Assay

The SH-SY5Y cells were seeded onto 96 well plates at cell density of 25,000 cells per well and incubated. After 24 h cells were treated with different concentration of compounds (10, 20, 50, 100 and 200 μM) and incubated. After 24 h MTT (5 mg/mL) was added to the cells and incubated for 3 h, removed media and DMSO/Methanol (1:1) solution added to dissolve formazan crystals. The absorbance was recorded at 570 nm and the percentage cell viability was calculated with respect to control cells treated with vehicle (DMSO).

### In Cellulo Antioxidant Assay

To assess in cellulo antioxidant activity of NMIs, rescue of neuronal cells from H₂O₂ induced cell death was assessed by MTT assay. The cells were seeded onto 96 well plate at seeding density of 25,000 cells per well. After 24 h cells were treated with H₂O₂ (200 μM) alone and with NMIs (20 μM) for 18 h. H₂O₂ treatment result in neuronal cell death and potent antioxidant molecule rescue cells. The cell viability was assessed by MTT assay and percentage cell viability was calculated.

Neuronal Cell Rescue from Aβ Toxicity

Cell rescue from Aβ toxicity by NMIs were assessed in SH-SY5Y cells. The cells were seeded into 96 well plate at cell density of 25,000 cells per well and after 24 h the media was replaced with DMEM-F12 with low serum (2.5% FBS). After 6 h, cells were treated with Aβ (10 μM) alone and with NMIs (20 μM) in low serum media. Different concentrations of compound (10, 20 and 50 μM) were used to treat cells. For metal dependent studies, Aβ (10 μM) with metals (20 μM) in the absence and presence of NMIs (20 μM) was treated. The cells were incubated for 30 h and MTT assay was performed to calculate the percentage cell viability.

### Mitochondrial Membrane Potential (MMP) Measurements

For quantifying relative change in MMP, SH-SY5Y cells were seeded into 96 plates and incubated. After 24 h, cells were treated with Aβ (20 μM) alone and with two different concentrations of NMIs (M3) (20 and 50 μM) for 18 h and stained with Mitotracker Orange (500 nM) in media for 15 min followed by washing with PBS twice. Then quickly fluorescence was measured (λₑₓ=554 nm and λₑₘ=576 nm) by Spectramax multi-well plate reader in well scan mode (>80 points, with 6 flashes per read) and averaged the value. The relative fluorescence of treatments in comparison with control was calculated to assess the change in MMP.

For fluorescence imaging, cells were seeded into glass bottom dishes and incubated for 24 h. Then treated with Aβ (20 μM) alone and with NMIs (M3) (50 μM) in low serum media for 18 h and stained with Mitotracker Orange (500 nM) in media for 15 min. Cells were washed with PBS thrice and imaged in live cell set up in Rhodamine channel using 63× oil immersion using Leica DMi8 fluorescence microscope.

### Mitochondrial ROS Measurements

For quantifying mitochondrial ROS levels, SH-SY5Y cells were seeded into 96 well plates and incubated. After 24 h cells were treated with Aβ (20 μM) alone and with two different concentrations of NMIs (M3) (20 and 50 μM) for 8 h and stained with MitoSox Red (5 μM) in PBS for 15 min followed by a wash with PBS thrice. Then quickly fluorescence was measured (λₑₓ=510 nm and λₑₘ=580 nm) by Spectramax multi-well plate reader in well scan mode (>80 points, with 6 flashes per read). The relative fluorescence was used to assess ROS levels with respect to control. For ROS imaging, cells were seeded onto glass bottom dishes and incubated for 24 h. Cells were treated with Aβ (20 μM) and with NMIs (M3) (50 μM) in low serum media for 8 h. Then the cells were washed with PBS and stained with MitoSOX (5 μM) prepared in PBS and incubated. After 15 min, wash thrice with PBS and imaged in live cell set up in rhodamine channel using 63× oil immersion using Leica DMi8 fluorescence microscope.

### Imaging Mitochondrial Structural Changes

The effect of NMIs (M3) on Aβ induced structural damage to mitochondria was assessed and Mito-TG probe was used for imaging mitochondria. SH-SY5Y cells were seeded onto confocal dishes and incubated for 24 h. Then cells were treated with Aβ (20 μM) alone and with M3 (50 μM) and incubated for 8 h. Followed by PBS washing, and stained with Mito-TG (250 nM) for 20 min. Cells were washed thrice with dPBS and imaged in live cell set up in rhodamine channel using 63× oil immersion using Leica DMi8 fluorescence microscope.

### Apoptosis Assay

The apoptosis induced by Aβ and rescue by NMIs (M3) was assessed through nuclear staining and morphology analysis. Apoptosis induces DNA damage and morphological change in the nucleus. Based on the nuclear features the extent of apoptosis was assessed as percentage apoptotic cells. SH-SY5Y cells were cultured and treated with Aβ (10 μM) and with NMIs (M3) (20 μM) to quantify the apoptosis and rescue by NMIs (M3). After 24 h the cells were washed with PBS and fixed with 4% paraformaldehyde and then with PBS for 5 min thrice and the nucleus was stained with DAPI (1 μM) for 10 min, washed thrice with PBS and imaged using fluorescence microscope to visualize the nuclear morphology. The cells exhibiting abnormal shape, chromatin condensation, nuclear shrinkage have been counted as apoptotic cell. Total of more than 300 cells were counted to calculated the percentage of apoptosis.

### Indirect Immunofluorescence

Cells were cultured in confocal dishes up to 70% confluence and then treated with Aβ alone and with NMIs (M3), and subjected to immunofluorescence. The cells were washed twice with dPBS and then fixed with 4% paraformaldehyde for 15 min. The cells were then washed twice with dPBS for 5 min followed by treatment with permeabilization buffer (dPBS with 0.1% Triton X-100) for 10 min. The permeabilization buffer was aspirated and rinses with dPBS twice for 5 min. The samples were blocked with 10% goat serum in dPBS for 30 min at room temperature. The blocking solution was aspirated and then was treated with primary antibody (Nrf2/Iba1) with desired dilution in 4° C. with gentle shaking overnight. The samples were then washed with dPBS thrice for 5 min each and treated with Alexa Fluor-488 conjugated secondary antibody for 1 h in room temperature. The cells were washed with dPBS thrice and counterstain with DAPI (1 μM) for 10 min and imaged using confocal microscope.

The negative control sample was prepared with a similar procedure without primary antibody treatment and found no/weak fluorescence signals in the samples.

### Bio-AFM Experiments

Microglial cells are part of the inherent defense mechanism against any pathological assault in the brain. These cells remain in a dormant surveillance state and are activated by the pathological stimuli to various state of reactivity. The microglial cells were known to constantly scan the brain and get activated encountering pathological stimuli and remove it. Microglia plays a role in the removal of amyloid aggregates and in the propagation of Aβ to the unaffected region. In the AD brain, over activation of microglia induced neuroinflammation and neuronal death mediated by inflammatory mediators. Thus the microglia acts as a double edged sword that exerted both beneficial and detrimental effects. Targeting microglial activation and neuroinflammation were tangible targets to develop therapeutics for AD. Aβ, excess ROS, oxidative stress and mitochondrial damage are the major factors directly or indirectly responsible for microglial activation.

Microglia activation and its alleviation was studied by Bio-AFM technique combining the indirect immunofluorescence by confocal microscopy and cellular mechanical properties characterization by AFM. C6 microglia was used as model cells and Aβ as pathological stimuli for the study. C6 cells were cultured in glass bottom confocal dishes to attain 70% confluence. The cells were primed to activation by using 500 ng/mL of LPS in low serum media (2.5% FBS). Then microglial cells were treated with 5 μM Aβ42 alone and with NMIs (M3) for 18 h. Then the cells were fixed and probed for Iba1 marker by indirect immunofluorescence, counter stained with Phalloidin rhodamine (200 nM) for actin labeling to visualize the actin distribution and DAPI (1 μM) for labeling the nucleus. The samples were washed thrice using dPBS and subjected to Bio-AFM imaging.

The Bio-AFM studies were performed in Bioscope Resolve AFM integrated with Olympus Fluoview-3000 Confocal laser scanning microscope (Bruker). The original confocal stage was removed and replaced with the Bruker Bio-AFM stage. For the confocal images aspect ratio was fixed to 4:3 with channel size of 640×480 and 3-point calibration was done to obtain proper overlay and pixel scaling was optimized by setting 0.375 μ/pixel. Region of interest was selected in the confocal using 60× oil immersion objective (Olympus plan Apo N) and on selected region AFM probe was landed using MIROVIEW window. Peak Force Quantitative Nanomechanical Mapping (PF-QNM) in Fluid mode was used for imaging the cells wherein this mode works based on Peakforce tapping principle. From this mode, height, log DMT and adhesion force of the samples was recorded. The log DMT was calculated using Derjaguin, Muller, Toropov (DMT) model using the formula given below. Studies were performed using MLCT BIO-F probe made up of non-conductive silicon nitride, coated with reflective Au with spring constant of 0.60 N/m and tip radius of 20 nm. Images were recorded with scanning speed of 0.5 Hz with 512 samples per line. The confocal images were recorded in DAPI, Alex488 and rhodamine channels. AFM data was analyzed using NanoScope analysis software (1.9 version) and confocal images were analyzed using Image J. Ftip=4/3E*√Rd3+Fadh, where Ftip=Force on tip, Fadh=Adhesion force, R=Tip radius and d=Tip sample separation.

### Western Blot Experiments

Western blot analysis was performed to analyse the changes in the protein levels from cell lysates. SH-SY5Y and C6 cells were cultured to 80% confluence in T25 flask, trypsinised and seed into 12 well plate. The cells were cultured for 30 h and then treated with LPS (500 ng/mL) in low serum media for 6 h. Then the cells were treated with Aβ42 (5 μM) alone and with NMIs (M3) (10 μM) for 18 h. The cells were harvested by trypsinisation and the protein was extracted in RIPA lysis buffer with protease inhibitor cocktail (1×). Briefly cells were washed with dPBS and lysed in 50 L of RIPA lysis buffer with gentle shaking in 4° C. for 30 min. Then the cells were centrifuged at 16000 g for 20 min and the supernatant containing protein was collected and quantified by Bradford assay. The protein lysate was subjected to SDS-PAGE (12% resolving gel) and transferred to PVDF membrane. The blot paper was blocked with 5% skim milk powder for 1 h at room temperature and then washed thrice with TBST. The blots were incubated with primary antibodies for 20 h at 4° C. The blots were washed thrice with TBST for 5 min and then incubated with anti-rabbit HRP-conjugated secondary antibodies for 1 h at room temperature. Finally, the blots were washed thrice using TBST and developed with ECL reagent and imaged in BioRad ChemiDoc instrument. All the western blots were quantified by using ImageJ, data was plotted and analysed by GraphPad prism5.

### Example 3

### Modulation of Aβ Aggregation

The ability of the NMIs, the compounds of the present disclosure M1-M4 to inhibit Aβ aggregation and dissolve the preformed fibrillar aggregates was evaluated by dot blot assay as explained in Example 2 above. Aβ (10 μM) was incubated alone and with NMIs (20 μM) in phosphate buffer saline (PBS, 10 mM and pH 7.4) at 37° C. for 48 h to form Aβ fibrillar aggregates. The samples were blotted and probed with fibril-selective OC antibody (rabbit anti-human amyloid fibrils). Dot blot data showed that M1 and M3 inhibited the formation of Aβ fibrils with ˜95% and 94% efficacy, respectively (FIG. 1A). Inhibition by M2 and M4 showed minimal effect with ˜11% inhibition. M3 was further assessed for inhibitory effect at lower concentrations.

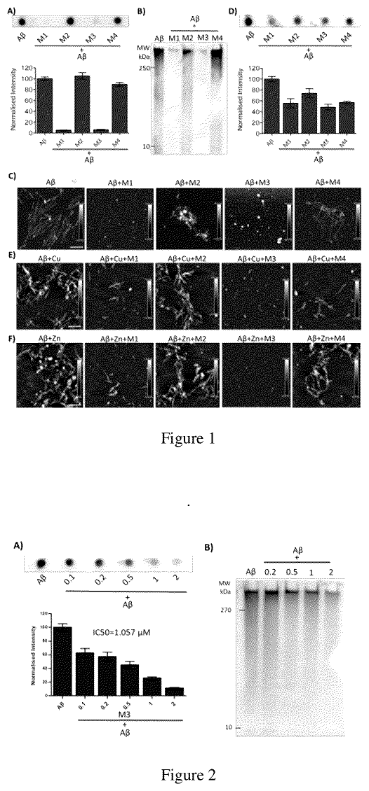

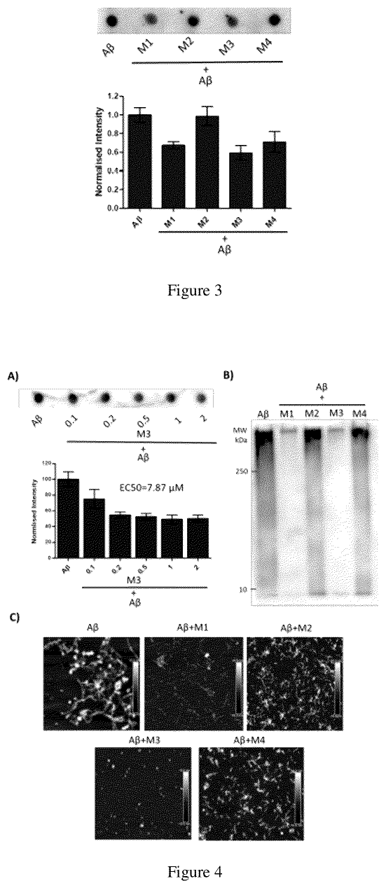

M3 showed concentration-dependent activity with 37%, 42%, 55%, 74% and 90% of inhibition at 0.1, 0.2, 0.5, 1 and 2 molar ratios with IC50 value of 1.0 μM (FIG. 2A). It has been observed that the oligomers are a highly toxic species compared to Aβ fibrillar aggregates. The ability of NMIs (M3) to inhibit the formation of Aβ oligomers was assessed by dot blot assay. Aβ peptide (10 μM) was incubated with NMIs at 4° C. for 24 h followed by probing with A11 oligomer-specific antibody. The blot data showed 33% and 40% oligomer inhibition by M1 and M2, respectively (FIG. 3). Native PAGE and western blot analysis was carried out to understand the size distribution of Aβ aggregated species. M1 and M3 treated samples showed a reduction of different Aβ species ranging from lower molecular weight (LMW) to higher molecular weight (HMW) species and specifically, M3 showed concentration dependent activity (FIGS. 1B and 2B). In order to analyse the higher order aggregates not permeable in the gel matrix and the morphological characteristics, Aβ samples were subjected to atomic force microscopy (AFM) imaging. AFM micrographs showed fibrillar structures for Aβ sample, while amorphous structures were observed for M1 and M3 treated samples, which indicate effective inhibition of Aβ fibrillar aggregation (FIG. 1C). M2 and M4 treated Aβ samples showed fibrillar structures inferring their inability to inhibit fibrillar aggregation.

The advanced stage of AD is characterized by the accumulation of Aβ aggregates in the brain, which needs to be dissolved and cleared for effective therapy. Therefore small molecules capable of dissolving the preformed Aβ aggregates are expected to effectively reduce the amyloid burden. Hence the ability of NMIs of the present disclosure was evaluated to dissolve the preformed Aβ fibrillar aggregates.

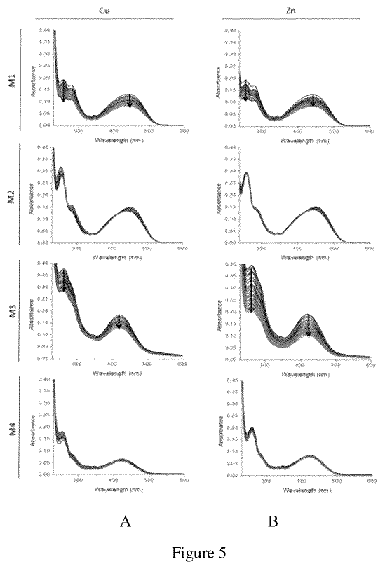

The fibrillar aggregates were formed by incubating Aβ peptide (10 μM) in PBS (pH 7.4) at 37° C. for 48 h. The aggregate samples were treated with NMIs (20 μM) for 48 h followed by dot blot analysis. M1-M4 showed ˜44%, 25%, 52% and 43% dissolution efficiency, respectively (FIG. 1D). M3 displayed concentration-dependent activity of 25%, 46%, 48%, 50% and 59% dissolution at 0.1, 0.2, 0.5, 1 and 2 molar ratio, respectively, with EC50 of 7.87 μM (FIG. 4A). The gel electrophoresis and western blot analysis have shown that M1 and M3 effectively inhibited the formation of LMW and HMW Aβ aggregates (FIG. 4B). Further, AFM imaging of M3 treated samples revealed amorphous clumps confirming the dissolution of fibrillar species (FIG. 4C). These studies confirmed that M3 as the potential candidate to dissolve Aβ fibrillar aggregates to possibly nontoxic species. The structural and functional integration of all three components viz., dopamine, DPA (di-2-picolylamine) and NMI core and their synergistic action made M3 a superior modulator of Aβ aggregation.

### Metal Chelation and Modulation of Metal-Dependent Aβ Aggregation

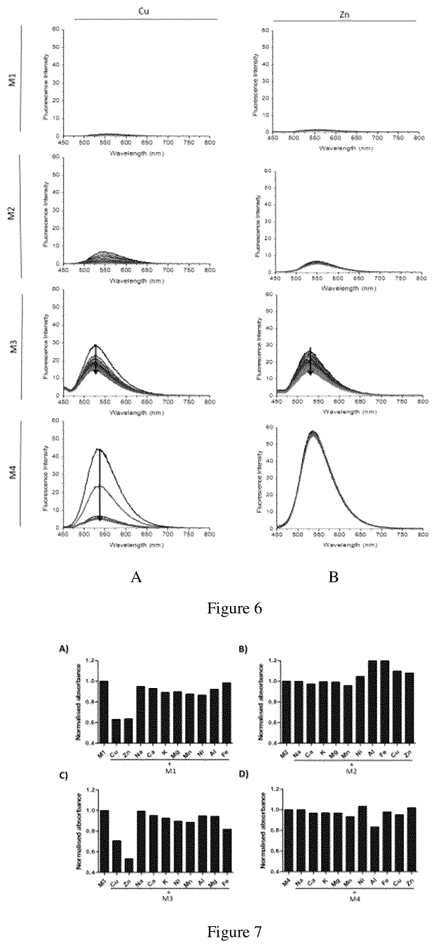

The compounds of the present disclosure (NMIs) were designed to chelate with metals (Cu and Zn) and alleviate the metal-dependent Aβ toxicity. UV-visible spectra of NMIs of the present disclosure showed two absorbance maxima at 265 nm and 420/440 nm. The fluorescence emission in water was negligible for M1 and M2, while M3 and M4 exhibited weak emission at 525 nm and 535 nm, respectively. The change in absorbance and fluorescence spectra of NMIs were monitored to understand the interaction with Cu(II) and Zn(II). The concentration-dependent hypochromic shift in the absorbance spectra showed that M1 and M3 effectively interacted with Cu (II) and Zn (II). There was no significant change in the spectra of M2 and M4 in the presence of Cu (II) or Zn (II) (FIGS. 5(A & B)). The fluorescence intensity of M3 at 525 nm decreased with the increasing concentration of Cu(II) or Zn(II) (FIGS. 6(A & B)). The absorbance studies with different physiologically relevant metals revealed that M1 and M3 selectively chelated with Cu(II) and Zn (II) (FIG. 7 (A-D)). The chelation of M1 and M3 with Cu (II) and Zn (II) was further supported by MALDI-mass spectrometry analysis, which showed m/z peaks at 377.234, 441.22, 477.12 and 531.04, 593.17, 630.38 corresponding to [M+H], [M+Cu] and [M+ZnCl] for M1 and M3, respectively.

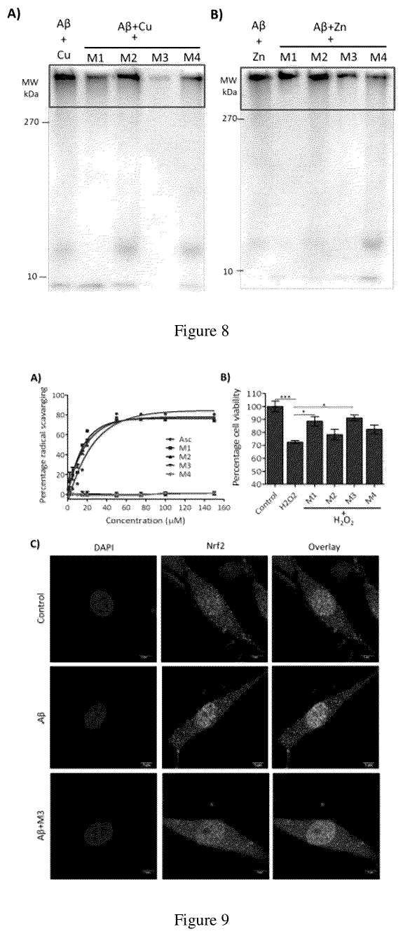

The absorbance and fluorescence spectroscopy studies together with mass spectrometry data confirmed the selective Cu (II) or Zn (II) chelation property of M1 and M3. Metal chelation and Aβ aggregation modulation activity of M1 and M3 was anticipated to synergistically inhibit metal-dependent Aβ aggregation. Aβ (10 μM) was incubated in the presence of Cu (II) or Zn (II) (20 μM) with NMIs at 37° C. for 48 h and the samples were subjected to native PAGE and western blot analysis. The metal ions were known to enhance Aβ aggregation and the western blot results showed the formation of higher order Aβ aggregates in the presence of Cu (II) and Zn (II) (FIGS. 8(A & B)). Aβ samples incubated with M1 and M3 reduced the formation of aggregates in the presence of Cu (II), while M3 and M4 were found to reduce Zn-dependent Aβ aggregation. Modulation of Zn-dependent Aβ aggregation by M4 was attributed to its two Zn (II) chelation centres. M3 with dopamine and DPA moieties effectively modulated both Cu (II) and Zn (II)-dependent Aβ aggregation. The effect of NMIs on the morphological outcome of metal-dependent Aβ aggregation was visualized by AFM imaging (FIGS. 1E and 1F). Aβ in the presence of Cu (II) and Zn (II) formed dense and thick fibrils. In the presence of M3, minimal Aβ fibrils and mostly small amorphous clumps were observed.

All the above results from spectroscopy, western blot and AFM imaging established M3 as a selective Cu (II) and Zn (II) chelator, which effectively inhibited metal-dependent Aβ aggregation. Dopamine and DPA pharmacophores chelated metals (Cu (II) and Zn (II)), while NMI core synergistically inhibited metal-dependent Aβ aggregation.

### Modulation of ROS and Oxidative Stress

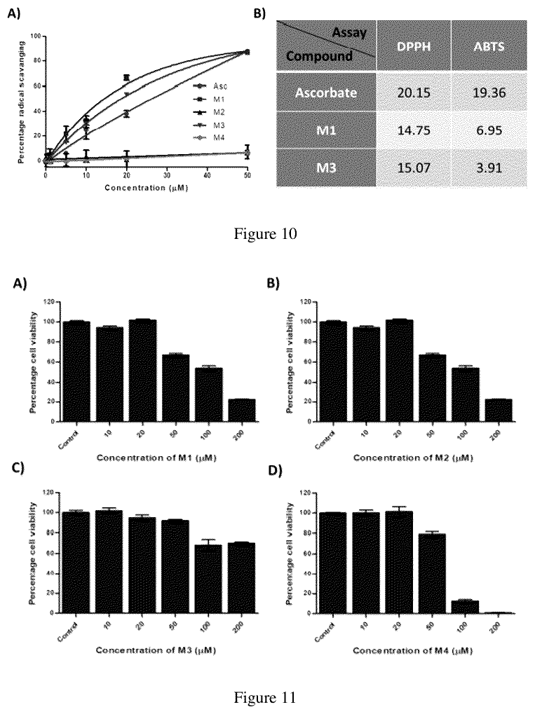

Excess production of reactive intermediate species (ROS and RNS: reactive nitrogen species) and associated oxidative stress, biomolecular and mitochondrial damage, and neuroinflammation are the additional traits of amyloid toxicity. NMIs of the present disclosure are equipped with DPA and dopamine to synergistically modulate metal-dependent and independent ROS generation, oxidative stress, and related pathological events. In vitro 2, 2-diphenyl-1-picrylhydrazyl (DPPH) and 2, 2′-azino-bis(3-ethylbenzothiazoline-6-sulfonic acid) (ABTS) radical scavenging assays were performed to assess the ROS scavenging ability of NMIs. The stable DPPH radical in methanol absorbed at λₘₐₓ 517 nm and quenching of radicals by antioxidants caused a decrease in absorbance intensity. The percentage radical quenching was calculated to assess the antioxidant capacity of compounds under study in comparison with a standard (ascorbate, a natural antioxidant). The results from DPPH radical scavenging assay showed that M1 and M3 effectively scavenge DPPH radicals with an EC50 value of 14.75 and 15.07 μM, respectively, which were better than ascorbate with EC50 of 20.15 μM (FIGS. 9A and 10B).

ABTS (λₘₐₓ at 734 nm) radical scavenging assay assessed the ability of NMIs to quench nitrogen radicals (RNS). Compounds capable of quenching ABTS radically reduced the absorbance intensity at 734 nm. M1 and M3 showed superior ABTS radical quenching ability with EC50 of 6.95 μM and 3.91 μM, respectively, outperforming ascorbate with EC50 of 19.36 μM (FIGS. 10A and B). These in vitro experiments have established M1 and M3 as effective antioxidants and further their ability to scavenge ROS in cellulo was assessed. Treatment of cells with H₂O₂ was known to generate ROS and cause DNA damage, protein oxidation, lipid peroxidation and mitochondrial dysfunction, which led to cell death. The neuronal cells were treated with H₂O₂ (200 μM) alone and with NMIs (20 μM) for 18 h and cell viability by MTT assay. M1 and M3 significantly rescued cell death from ROS and oxidative stress with cell viability of 89% and 91%, respectively in comparison to H₂O₂ treated cells (˜72%) (FIG. 9B). The in vitro and cellular studies demonstrated that M3 as the best antioxidant and ROS modulator. The production of excess ROS by Aβ results in oxidative stress and elicits stress response.

M3 with in vitro antioxidant activity and in cellulo ROS quenching effect was expected to alleviate oxidative stress and modulate Nrf2 response. The effect of M3 on Nrf2 localisation in Aβ treated SH-SY5Y cells were assessed by indirect immunofluorescence staining study. The cells treated with Aβ developed oxidative stress and as a stress response Nrf2 translocate and accumulate more in the nucleus as compared to control cells (FIG. 9C). M3 treatment quenched the ROS and reduced oxidative stress, which in turn reduced the translocation of Nrf2 to the nucleus.

Rescue of Neuronal Cells from Metal-Independent and Dependent Aβ Toxicity

NMIs modulated metal-independent and dependent Aβ aggregation, reduced ROS in vitro and in cellulo, and rescue cells from oxidative stress. The biocompatibility, the cytotoxicity of compounds of the present disclosure to neuronal cells were evaluated by MTT assay. SH-SY5Y cells were treated with different concentrations of modulators (NMIs) for 24 h and performed MTT assay to determine the cell viability. The results showed that NMIs were non-toxic to cells over a concentration range up to 20 μM. Specifically, M3 was found to be biocompatible with minimal toxicity up to 200 μM (FIGS. 11 A-D).

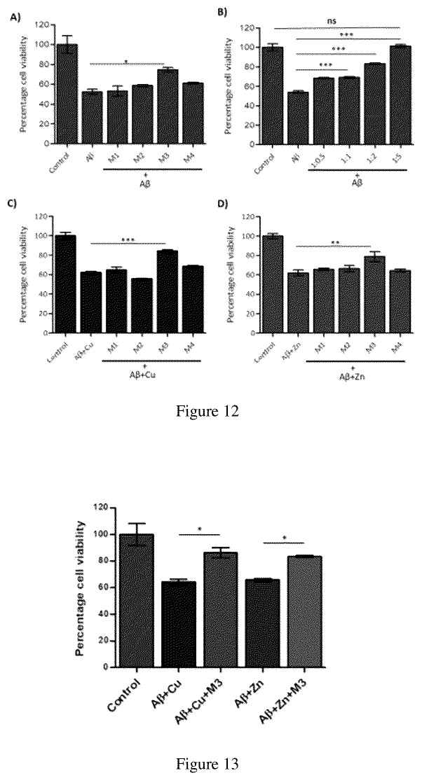

Compounds of the present disclosure (M1 to M4) were evaluated for their ability to rescue neuronal cells from Aβ toxicity by MTT assay. Aβ treatment induced toxicity to neuronal cells through many pathways and reduced the cell viability to ˜52%. The cells incubated with Aβ in the presence of M3 showed significant improvement in cell viability to ˜75%. whereas other NMIs showed minimal effect to alleviate Aβ toxicity (FIG. 12A). M1 showed lowered alleviation of the Aβ toxicity in the cellular milieu in spite of showing in vitro aggregation modulation and antioxidant property, which was possibly attributed to its low bioavailability and degradation.

Further, the effect of concentration of compounds of the present disclosure on the cell rescue was assessed. The results showed concentration-dependent effect with cell viability increased to 68%, 70%, 83% and near-complete rescue at 5, 10, 20 and 50 μM concentrations of M3, respectively, compared to control (54%) (FIG. 12B). M3 at a low concentration of 5 μM exhibited significant rescue of cells from Aβ toxicity, while the complete rescue was observed for a higher concentration of 50 μM. The ability of NMIs to rescue cells from metal-dependent Aβ toxicity were assessed in the presence of Cu (II) and Zn (II). Cells were treated with Aβ (10 μM) along with metal ions (20 μM) and NMIs (20 μM) for 30 h. Aβ in the presence of Cu (II) reduced cell viability to ˜62% and M3 treatment had rescued cells with viability to 85% (FIG. 12C). Aβ with Zn (II) viability reduced to ˜61% and M3 treatment rescued cells by improving the viability to ˜79% (FIG. 12D). At a higher concentration of M3 (50 M), metal-dependent Aβ toxicity was combated with significantly enhanced viability of 86% and 83% for Cu (II) and Zn (II), respectively (FIG. 13). These in-cellulo experiments confirmed the ability of M3 to effectively rescue neuronal cells from metal-independent and dependent Aβ toxicity.

### Modulation of Mitochondrial Damage and Neuronal Apoptosis

Aβ interaction with mitochondria instigated the generation of excess ROS, impairment of oxidative phosphorylation, loss of mitochondrial potential and ATP synthesis, and cell death mediated by various pathways that are triggered by mitochondrial damage.

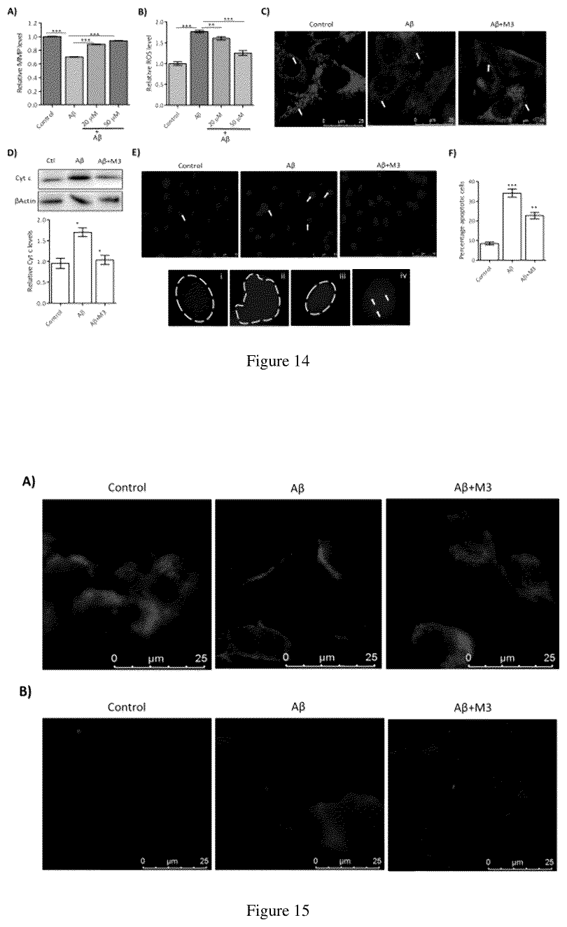

The compound M3 with aggregation modulation and antioxidant activity potentially prevent Aβ mediated mitochondrial damage. Quantitatively measurement of mitochondrial membrane potential (MMP) changes was carried out. SH-SY5Y cells were cultured in 96 well plate, treated with Aβ (20 μM) alone and with different concentrations of M3 (20 μM and 50 μM) for 18 h and relative MMP was measured using MitoOrange fluorescence, a MMP-dependent mitochondrial probe. MMP of Aβ treated cells dropped to 70% compared to control (100%) reflecting the functional damage to mitochondria. Upon treatment with M3 at two concentrations 20 μM and 50 M, MMP was improved to 88% and 93%, respectively, which indicated prevention of mitochondrial damage (FIG. 14A).

Changes in MMP were visualized by treating the cells with Aβ (20 μM) alone and with M3 (50 μM) for 18 h followed by staining with MitoOrange. The fluorescence imaging showed a decrease of MMP in cells incubated with Aβ, while MMP was least affected in the presence of M3 (FIG. 15A). Aβ was known to interact with mitochondria and increase the ROS levels, which resulted in oxidative damage to neuronal cells. The ability of M3 to modulate Aβ-induced ROS levels in mitochondria was evaluated by the MitoSOX probe. The cells were treated with Aβ (40 μM) alone and with two concentrations of M3 for 8 h followed by treatment with MitoSOX to measure the relative ROS levels. Aβ has elevated the ROS levels to 1.76 fold compared to control cells. The mitochondrial ROS levels reduced to 1.6 and 1.25 fold upon treatment with 20 and 50 μM of M3, respectively (FIGS. 14B and 15B).

Aβ-associated functional abnormalities induced structural defects in mitochondria. The effect of M3 to prevent structural damage was assessed by staining the cells with Mito-TG, a mitochondrial probe. The cells were treated with Aβ (20 μM) alone and with M3 for 18 h, followed by Mito-TG staining and fluorescence microscopic imaging to visualize the structural changes. The Aβ treatment deformed mitochondria with fragmented morphology in comparison to the long tubular structure of healthy cells (FIG. 14C). M3 treatment protected normal long tubular structures of cells from Aβ-induced deformation and fragmentation. Collectively, these structural and functional studies of mitochondria confirmed that M3 is a potential modulator of Aβ-induced toxicity to protect mitochondrial damage. The plausible mechanism involved inhibition of pathological Aβ interaction with mitochondria and scavenging of ROS by M3, which synergistically prevented mitochondrial damage.

Mitochondrial damage by Aβ was known to increase the level of cytochrome c (Cyt c) and induce neuronal apoptosis. The effect of M3 on Cyt c levels in cells was evaluated under Aβ-induced toxicity conditions. Neuronal cells were treated with Aβ (10 μM) alone and with M3 (20 μM), Cyt c levels were assessed by western blot analysis. Cyt c levels in Aβ treated cells increased significantly by 1.7 fold compared to control (FIG. 14D). In cells treated with Aβ and M3, Cyt c levels were found to be similar to control cells, which suggested the protective role of M3 against Aβ-induced mitochondrial damage. The reduction of Cyt c levels and protection of mitochondria by M3 in turn contributed to the neuronal rescue by modulating the apoptosis pathway.

Modulation of apoptosis by M3 was quantified by apoptosis assay through nuclear staining and morphological analysis. Apoptotic signals induced nuclear DNA damage show abnormal nuclear features, which has been used to evaluate and quantify the extent of apoptosis. Neuronal cells treated with Aβ (10 μM) alone and with M3 (20 M) for 24 h were stained for the nucleus and analysed for apoptotic morphological features. The fluorescence microscopy images showed Aβ induced apoptosis as revealed by the damaged and distorted nucleus, nuclear shrinkage and condensation of chromatin compared to the normal nucleus in healthy cells (FIGS. 14E, inset i to iv). M3 treated cells showed normal nuclear structures, which was an indication of cellular rescue from apoptosis. Quantification data revealed that Aβ induces a significant level of apoptosis (˜35%) as compared to control cells with ˜8.5% apoptosis (FIG. 14F). M3 treatment had significantly reduced apoptosis of neuronal cells to 22.5%. Overall, these studies demonstrated that M3 rescues neuronal cells from mitochondrial damage and Cyt c mediated apoptosis.

### Modulation of Microglial Activation

Bio-AFM was used to characterize molecular, morphological and mechanical properties of activated microglial and its modulation by multifunctional modulator M3. Microglia upon activation tends to change morphology accompanied by significant changes in the cytoskeletal network and mechanical properties. Indirect immunofluorescence imaging for molecular characterization and AFM for morphological and mechanical analysis using an integrated Bio-AFM instrumentation was used.

The protein marker Iba1 was imaged for microglia activation, actin for the cytoskeletal network using confocal microscopy, and the morphological and mechanical changes viz., shape, height, stiffness and adhesive forces were assessed using AFM. This enabled analysis of the microglial activation and the effect of M3 by correlating molecular level events and mechanical properties.

C6 microglial cells were used as model and Aβ as stimuli for microglial activation that mimics AD conditions. The C6 cells were primed with LPS (500 ng/mL) for 4 h followed by treatment with Aβ (5 μM) alone and with M3 (10 μM) for 18 h. The cells were stained for Iba1, actin (phalloidin-rhodamine) and nucleus (DAPI), and the samples were subjected to confocal and AFM imaging using integrated Bio-AFM instrument.

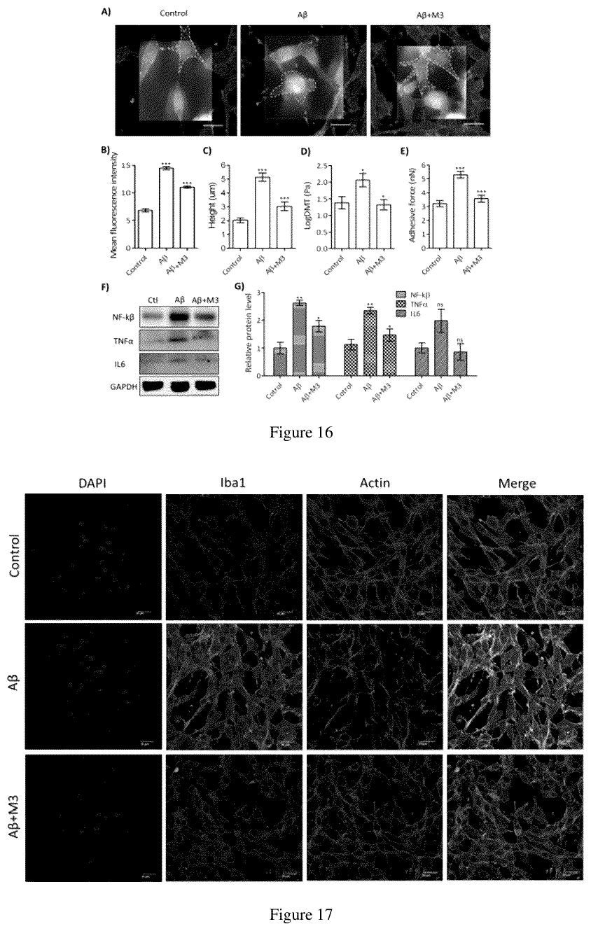

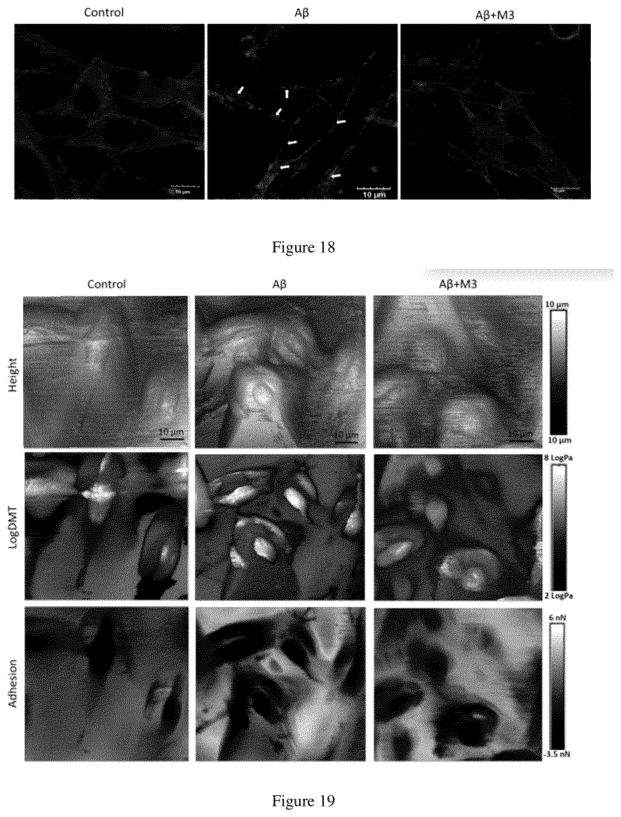

The Bio-AFM data showed activation of microglial cells as reflected by overexpression of Iba1 in Aβ treated cells compared to control (FIGS. 16A and 17). In the presence of M3, the Iba1 level has reduced significantly as compared to Aβ treated cells indicating reduction or prevention of microglial activation (FIG. 16B). In activated microglia, Iba1 interacts with actin filaments and potentially assists cytoskeletal rearrangements by crosslinking actin filaments, which was evident in the overlay fluorescence image of Aβ treated cells (FIG. 17). M3 treatment has reduced the Iba1 localisation with actin suggesting the reduction of crosslinking and remodelling of actin. Activation of microglial cells resulted in actin redistribution to the periphery and forms bundling to reform uropod, filopodia and lamellipodia structures. The actin labelling had clearly shown its redistribution to the periphery of Aβ treated microglial cells and was reduced by M3 (FIG. 18). These data clearly suggested that Aβ activates microglial and associated molecular events and M3 treatment effectively reduce microglial activation to healthy state.

The physical and mechanical parameters like height, stiffness and adhesive force by Peakforce Quantitative Nanomechanical mapping (PF-QNM) AFM imaging (FIG. 19). The height of activated microglial cells had increased to ˜5.13±0.29 m from ˜2.03±0.18 m of control cells and M3 treatment rescued cells from activation by preserving the height to ˜3.03±0.32 m (FIG. 16C). These height profiles suggested that the microglial activation induces bulge and amoeboid morphology, which was effectively rescued by the modulator M3.

Activation of microglia cells resulted in the change of cellular mechanical properties like stiffness and adhesive force due to actin remodelling and rearrangements in the cytoskeleton. The cellular stiffness was assessed by determining the log of Derjaguin, Muller, Toropov (Log DMT) modulus as a measure of Young's modulus. The control cells exhibited log DMT of ˜1.38±0.19 Pa, which increased to ˜2.06±0.20 Pa for Aβ treated microglia. This indicated a significant increase in cellular stiffness of activated microglia. M3 treatment reduced the log DMT value of Aβ treated microglia to ˜1.32±0.15 indicating reversal of cellular stiffness to healthy state (FIG. 16D). The activation of microglia, overexpression of Iba1, and actin remodelling enhanced the membrane ruffling that resulted in a change in adhesiveness of the cellular surface. The adhesive force of activated microglial cells had increased to ˜5.30±0.24 nN from 3.21±0.23 nN (control) and M3 treatment reduced to ˜3.57±0.25 nN, which is comparable to control cells (FIG. 16E). These changes in the molecular, mechanical, and structural properties characterized by Bio-AFM suggested the activation of microglial cells by Aβ and rescue by the multifunctional modulator M3. Effective modulation of microglial activation underscores the potential of M3 as a therapeutic candidate for AD and other neurodegenerative and neuroinflammatory disorders.

### Modulation of Neuroinflammation

Activation of microglia trigger neuroinflammation by releasing the inflammatory mediators which damage the neuronal function causing neurodegeneration. The Bio-AFM study clearly demonstrated the potential of M3 in reducing microglial activation. M3 can further reduce neuroinflammation by decreasing the inflammatory mediators by microglial cells. Aβ activates microglial cells and induce neuroinflammation mediated by NF-kβ pathway.

NF-kβ is a tangible drug target for neuroinflammation and AD. M3 with catechol moiety was expected to reduce neuroinflammation by suppressing NF-kβ pathway. C₆ microglia were primed with LPS (500 ng/mL) followed by Aβ (5 μM) treatment to induce inflammation, which was monitored by the change in NF-kβ levels in western blot analysis (FIG. 16F). Aβ treated cells showed 2.62 fold enhancement in NF-kβ level in comparison to control cells, which confirmed the neuroinflammation. M3 treatment had significantly downregulated the levels of NF-kβ to 1.78 fold compared to control (FIG. 16G).

Activation of NF-kβ resulted in upregulation of inflammatory mediators like TNFα, IL6 and IL1β that mediate neuroinflammation in AD. Quantification from western blot analysis showed 2.35 fold increase of TNFα level in Aβ treated cells in comparison to control and as anticipated M3 has reduced to 1.47 fold (FIG. 16G). Similarly, IL6 level was increased in Aβ treated compared to control and was reduced by M3, while statistical analysis showed that the changes were insignificant. The inflammatory mediators are known to induce neuronal damage through various pathways like apoptosis and necroptosis, and further enhanced microglial activation.

The synergistic action of M3 to prevent apoptosis and modulate TNFα levels also curbed neurodegeneration. The modulation of neuroinflammatory mediators had revealed the protective effect of M3 and its potential as a therapeutic candidate for AD and other neurodegenerative and neuroinflammatory disorders.

The multifunctional molecules with multiple functional pharmacophores of the compounds of the present disclosure synergistically tackled multifaceted AD pathology. In specific the compound M3 is a multifunctional modulator targeting major pathological factors viz, Aβ aggregation, metal-Aβ toxicity, oxidative stress, mitochondrial damage, microglial activation and neuroinflammation. Dot blot, western blot and AFM data showed effective modulation of Aβ aggregation. The in vitro antioxidant assays and cellular studies demonstrated the modulation of ROS and oxidative stress. The compound M3 reduced Cyt c levels, protect mitochondria, and rescued neuronal cells from apoptotic cell death. The detailed Bio-AFM studies and molecular characterization validated the protective effect of compounds of the present disclosure by preventing microglial activation and associated adverse molecular events and NF-kβ mediated neuroinflammation. Thus the compounds of the present disclosure are multifunctional modulator to synergistically ameliorate the multifaceted toxicity of AD, and other neurodegenerative and neuroinflammatory disorders.

### Advantages of the Present Disclosure

The above-mentioned implementation examples as described on this subject matter and its equivalent thereof have many advantages, including those which are described.

Small molecule-based naphthalene monoimide (NMI) compounds of Formula (I) of the present disclosure synthesized through specific design strategy and pharmacophores provides maximum inhibition of Aβ42 fibrillar assembly involved in amyloidogenesis and rescue of neuronal cells from amyloid toxicity. The compounds of Formula (I), its stereoisomers or a pharmaceutically acceptable salt thereof possesses improved hydrophobicity and exhibit enhanced capability to penetrate the plasma membranes of live cells. The compounds of Formula (I), its stereoisomers or a pharmaceutically acceptable salt thereof of the present disclosure modulates aggregation of Aβ42, tau, ca-syn, and amylin and helps in treating condition or disorder or diseases mediated by aggregation of Aβ42, tau, α-syn, and amylin. Additionally, the compounds of the present disclosure also alleviate metal dependent amyloid toxicity. Further, the compounds of the present disclosure clearly establishes a multifaceted therapeutic effect as it is capable of synergistically modulating metal independent and dependent amyloid toxicity, scavenge reactive oxidative species (ROS), alleviate oxidative stress, emulate Nrf2 mediated stress response, mitochondrial damage, microglial activation and neuroinflammation. The compounds of the present disclosure are also found to suppresses NF-kβ.

This confirmed that the compounds of the present disclosure and their pharmaceutical composition are efficient candidates for treatment of a condition or a disease mediated by amyloid toxicity. The compounds and their pharmaceutically acceptable salts thereof can be used for effective treatment of conditions mediated by neurodegenerative diseases such as Alzheimer's disease (AD), Parkinson's disease (PD), prion diseases, polyglutamine expansion diseases, Huntington's disease (HD), tauopathies, frontotemporal dementia associated with tau-immunoreactive inclusions (FTD-tau), progressive supranuclear palsy (PSP), corticobasal degeneration (CBD), amyotrophic lateral sclerosis (ALS), lewibody disease, and spinal muscular atrophy. Moreover, the present disclosure also provides a simple synthetic routes to prepare the compounds which can be manufactured in large-scale industrially. Further, the present disclosure also provides pharmaceutical composition comprising the compounds of Formula (I) along with other clinically relevant modulators and pharmaceutical carriers can be administered in effective amounts to treat both moderate and advanced stages of neurodegenerative diseases. Further the compounds of Formula (I) are capable of providing reversal of cognitive decline or improvement of cognitive decline.

1. A compound of Formula (I), 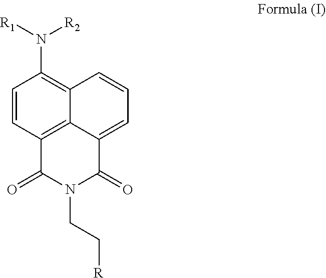
a stereoisomer thereof, or a pharmaceutically acceptable salt thereof;
wherein:
R is selected from C₆₋₁₀ aryl, C₅₋₁₈ heteroaryl, or NR₃R₄, wherein C₆₋₁₀ aryl is optionally substituted with two or more hydroxyl groups;
R₁ and R₂ are independently selected from C₁₋₆ alkyl, C₂₋₆ alkenyl, or C₂₋₆ alkynyl, wherein C₁₋₆ alkyl is optionally substituted with one or more groups selected from C₆₋₁₀ aryl, C₁₋₁₈ heteroalkyl, C₅₋₁₈ heteroaryl, C₃₋₁₂ cycloalkyl, or C₃₋₁₂ heterocyclyl; and
R₃ and R₄ are independently C₁₋₆ alkyl, wherein C₁₋₆ alkyl is optionally substituted with one or more groups selected from C₆₋₁₀ aryl, C₁₋₁₈ heteroalkyl, C₅₋₁₈ heteroaryl, C₃₋₁₂ cycloalkyl, or C₃₋₁₂ heterocyclyl.

2. The compound of Formula (I), the stereoisomer thereof, or the pharmaceutically acceptable salt thereof as claimed in claim 1; wherein
R is C₆₋₁₀ aryl substituted with two or more hydroxyl groups or NR₃R₄;
R₁ and R₂ are independently selected from C₁₋₆ alkyl, C₂₋₆ alkenyl, or C₂₋₆ alkynyl, wherein C₁₋₆ alkyl is optionally substituted with one or more groups selected from C₆₋₁₀ aryl, C₁₋₁₈ heteroalkyl, C₅₋₁₈ heteroaryl, C₃₋₁₂ cycloalkyl, or C₃₋₁₂ heterocyclyl; and
R₃ and R₄ are independently C₁₋₆ alkyl, wherein C₁₋₆ alkyl is optionally substituted with one or more groups selected from C₆₋₁₀ aryl, C₁₋₁₈ heteroalkyl, C₅₋₁₈ heteroaryl, C₃₋₁₂ cycloalkyl, or C₃₋₁₂ heterocyclyl.

3. The compound of Formula (I), the stereoisomer thereof, or the pharmaceutically acceptable salt thereof as claimed in claim 1; wherein
R is C₆₋₁₀ aryl substituted with two or more hydroxyl groups or NR₃R₄;
R₁ and R₂ are independently C₁₋₆ alky, wherein C₁₋₆ alkyl is optionally substituted with C₅₋₁₈ heteroaryl; and
R₃ and R₄ are independently C₁₋₆ alkyl, wherein C₁₋₆ alkyl is optionally substituted with C₅₋₁₈ heteroaryl.

4. The compound of Formula (I), the stereoisomer thereof, or the pharmaceutically acceptable salt thereof as claimed in claim 1; which is selected from the group consisting of:
a. 2-(3,4-dihydroxyphenethyl)-6-(dimethylamino)-1H-benzo[de]isoquinoline-1,3(2H)-dione;
b. 2-(2-(bis(pyridin-2-ylmethyl)amino)ethyl)-6-(dimethylamino)-1H-benzo[de]isoquinoline-1,3(2H)-dione;
c. 6-(bis(pyridin-2-ylmethyl)amino)-2-(3,4-dihydroxyphenethyl)-1H-benzo[de]isoquinoline-1,3(2H)-dione; and
d. 6-(bis(pyridin-2-ylmethyl)amino)-2-(2-(bis(pyridin-2-ylmethyl)amino)ethyl)-1H-benzo[de]isoquinoline-1,3(2H)-dione.

5. A process for preparation of a compound of Formula (I) a stereoisomers thereof, or a pharmaceutically acceptable salt thereof; 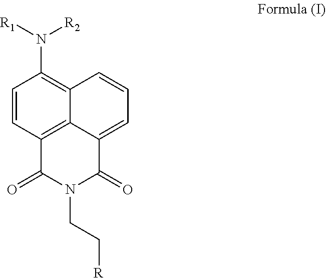
the process comprising: a) reacting a compound of Formula II with Formula III in the presence of a base and a solvent, to obtain a compound of Formula (I); 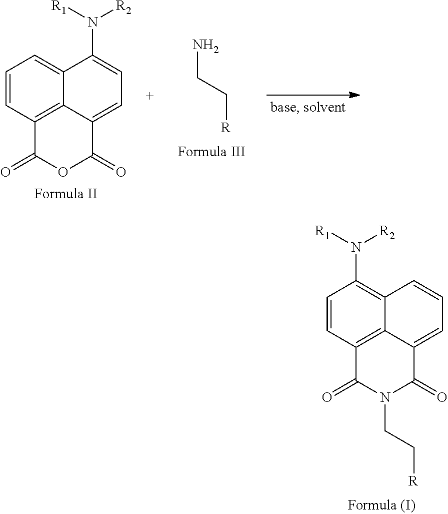
wherein:
R is selected from C₆₋₁₀ aryl, C₅₋₁₈ heteroaryl, or NR₃R₄, wherein C₆₋₁₀ aryl is optionally substituted with two or more hydroxyl groups;
R₁ and R₂ are independently selected from C₁₋₆ alkyl, C₂₋₆ alkenyl, or C₂₋₆ alkynyl, wherein C₁₋₆ alkyl is optionally substituted with one or more groups selected from C₆₋₁₀ aryl, C₁₋₁₈ heteroalkyl, C₅₋₁₈ heteroaryl, C₃₋₁₂ cycloalkyl, or C₃₋₁₂ heterocyclyl; and
R₃ and R₄ are independently selected from C₁₋₆ alkyl, wherein C₁₋₆ alkyl is optionally substituted with one or more groups selected from C₆₋₁₀ aryl, C₁₋₁₈ heteroalkyl, C₅₋₁₈ heteroaryl, C₃₋₁₂ cycloalkyl, or C₃₋₁₂ heterocyclyl.

6. The process as claimed in claim 5, wherein the base is selected from organic or inorganic bases selected from 1,8-diazabicyclo(5.4.0)undec-7-ene (DBU), N,N-diisopropylethylamine (DIPEA), triethylamine (Et₃N), C₁₋₁₀ alkyl amine, pyridine, or a combination thereof; and the solvent is selected from dimethyl formamide, ethanol, methanol, isopropyl alcohol, dimethylsulphoxide (DMSO), benzene, toluene, xylene, or a combination thereof.

7. The process as claimed in claim 5, wherein reacting the compound of Formula II with the compound of Formula III is carried out at a temperature in the range of 80 to 110° C. for a time period in the range of 2 to 5 hours.

8. The compound of Formula (I), the stereoisomer thereof, or the pharmaceutically acceptable salt thereof as claimed in claim 1; which reverses cognitive decline or improves cognitive decline.

9. A pharmaceutical composition comprising the compound of Formula (I) the stereoisomer thereof, or the pharmaceutically acceptable salt thereof as claimed in claim 1 with a pharmaceutically acceptable carrier, optionally in combination with one or more other pharmaceutical formulations.

10. The pharmaceutical composition as claimed in claim 9, wherein the composition is in a form selected from tablet, capsule, powder, syrup, solution, aerosol, or suspension.

11.-12. (canceled)

13. The compound of Formula (I), the stereoisomer thereof, or the pharmaceutically acceptable salt thereof as claimed in claim 1; which modulates aggregation of one or more proteins selected from the group consisting of Aβ, tau, α-syn, polyglutamine, and amylin (IAPP).

14. The compound of Formula (I), the stereoisomer thereof, or the pharmaceutically acceptable salt thereof as claimed in claim 1; which synergistically modulates metal independent and dependent amyloid toxicity, scavenges reactive oxidative species (ROS), alleviates oxidative stress, emulates Nrf2 mediated stress response, mitochondrial damage, microglial activation, and neuroinflammation.

15. The compound of Formula (I) as claimed in claim 1, the stereoisomer thereof, or the pharmaceutically acceptable salt thereof as claimed in claim 1; which suppresses NF-kβ.

16. A method for treatment of a condition mediated by a neurodegenerative disease in a subject, said method comprising administering to the subject an effective amount of the compound of Formula (I) the stereoisomer thereof, or the pharmaceutically acceptable salt thereof as claimed in claim 1; or a pharmaceutical composition comprising the compound of Formula (I), the stereoisomer thereof, or the pharmaceutically acceptable salt thereof with a pharmaceutically acceptable carrier, optionally in combination with one or more other pharmaceutical formulations.

17. A method for treatment of a condition mediated by a neurodegenerative disease or a neuroinflammation disorder in a subject, said method comprising administering to the subject a combination of (i) the compound of Formula (I), the stereoisomer thereof, or the pharmaceutically acceptable salt thereof as claimed in claim 1; or a pharmaceutical composition comprising the compound of Formula (I), the stereoisomer thereof, or the pharmaceutically acceptable salt thereof and a pharmaceutically acceptable carrier, optionally in combination with one or more other pharmaceutical formulations; and (ii) other clinically relevant immune modulator agents.

18. The method as claimed in claim 17, wherein the disease or disorder is selected from the group consisting of Alzheimer's disease (AD), Parkinson's disease (PD), prion diseases, polyglutamine expansion diseases, Huntington's disease (HD), tauopathies, frontotemporal dementia associated with tau-immunoreactive inclusions (FTD-tau), progressive supranuclear palsy (PSP), corticobasal degeneration (CBD), amyotrophic lateral sclerosis (ALS), Lewy body disease, and spinal muscular atrophy.

19. The method as claimed in claim 16, wherein the compound of Formula (I), the stereoisomer thereof, or the pharmaceutically acceptable salt thereof synergistically modulates metal independent and dependent amyloid toxicity, scavenges reaction oxygen species (ROS), alleviates oxidative stress, emulates Nrf2 mediated stress response, mitochondrial damage, microglial activation, and neuroinflammation.

20.-21. (canceled)

22. The method as claimed in claim 16, wherein the compound of Formula (I), the stereoisomer thereof, or the pharmaceutically acceptable salt thereof modulates aggregation of one or more proteins selected from the group consisting of Aβ, tau, α-syn, polyglutamine, and amylin (IAPP).

23. The method as claimed in claim 16, wherein the compound of Formula (I), the stereoisomer thereof, or the pharmaceutically acceptable salt thereof suppresses NF-kβ.

24. The method as claimed in claim 16, wherein the neurodegenerative disease is selected from the group consisting of Alzheimer's disease (AD), Parkinson's disease (PD), prion diseases, polyglutamine expansion diseases, Huntington's disease (HD), tauopathies, frontotemporal dementia associated with tau-immunoreactive inclusions (FTD-tau), progressive supranuclear palsy (PSP), corticobasal degeneration (CBD), amyotrophic lateral sclerosis (ALS), Lewy body disease, and spinal muscular atrophy.
